(19) United States
(12) Patent Application Publication
(10) Pub. No.: US 2007/0225781 A1
(43) Pub. Date: Sep. 27, 2007

(54) Apparatus and methods for altering temperature in a region within the body

(75) Inventors: Vahid Saadat, Saratoga, CA (US); Lorns G. Eltherington, Los Altos Hills, CA (US)

Correspondence Address: LEVINE BAGADE HAN LLP, 2483 EAST BAYSHORE ROAD, SUITE 100, PALO ALTO, CA, 94303, US

(73) Assignee: Nidus Medical, LLC, Saratoga, CA (US)

(21) Appl. No.: 11/386,239

(22) Filed: Mar. 21, 2006

(51) Int. Cl. A61F 7/00

(52) U.S. Cl. 607/105; 607/96

(57) ABSTRACT

Apparatus and methods for cooling and/or heating selected regions within a body are described herein. An implantable system is used to cool or heat nerve bodies down to about 15° C. to diminish nerve impulses. In one embodiment, the system can include an implantable unit containing a pumping mechanism and/or various control electronics. The system has a cooling element. The cooling element can be a Peltier junction or a catheter through which hot or cold fluid flows. The heated portion of the Peltier junction can be cooled by a liquid heat transfer medium which absorbs the heat from the junction and dissipates the heat elsewhere.

### TECHNICAL FIELD OF THE INVENTION

Apparatus and methods for temperature control of selected regions within a body are disclosed. The temperature control can be used to heat or cool, for several purposes including the control of pain and the treatment of chronic disease. Specifically, the apparatus and methods disclosed can be used to cool nerves, such as the spinal cord (e.g., dorsal and/or ventral columns), vagus nerves, femoral nerve, or sciatic nerve by implantable apparatus to impair conduction, or to heat the nerves to cause stimulation of the nerves. Additionally, tissues of the target organ(s) or muscles can be cooled or heated directly to offer further control of the impairment or activation of the organ.

### BACKGROUND OF THE INVENTION

It is generally known that cooling an injured region of the body typically helps to abate the associated pain. For example, cooling painful joints, inflamed tissue, or burned areas of skin can help with reducing the pain and inflammation. However, this type of treatment is generally limited to cooling via the surface of the skin, e.g., by applying a cold compress or an ice-pack.

Other methods of pain management include the use of analgesic antidepressant, anti-inflammatory, neuropathetic, antispasmotic and anxioilytic medications by multiple routes of administration. Invasive procedures such as nerve blocks, nerve destruction and nerve nerve stimulators are also widely used. Cognitive-behavioral therapy is also indicated in most chronic pain patients.

Such conditions, e.g., muscle spasms, may be painful, violent, and involuntary and affect a large segment of the population. This type of pain is often also chronic, i.e., lasts for one day or longer. Other conditions may result from injury or trauma to affected region within the body, such as to the muscles or to the nerves that innervate the muscles.

Examples of other painful conditions include sciatica and tendonitis. Sciatica is a condition characterized by pain radiating from the muscles in the back into the buttocks and may be a result of trauma to the spinal cord or to the sciatic nerve.

The debilitating effects of chronic pain are not only a source of anxiety and distress for the individual, but also represent a tremendous cost to society. For instance, workers suffering from chronic pain are frequently absent from work for weeks or even longer. This poses a great expense not only to the employer in sick-time coverage and disability pay, but also to society in lost productivity.

A variety of medicines are typically used in an attempt to alleviate the conditions associated with chronic pain. These have included muscle relaxants, such as methocarbamol, carisoprodol, mephenesin, etc. Nonsteroidal anti-inflammatory agents, such as ibuprofen, aspirin, and indomethacin are also used in conjunction with muscle relaxants for treating muscle spasms, tendonitis and sciatica. However, these methods provide, at most, partial relief and do not provide the type of relief considered adequate by most people. Accordingly, there exists a need for a method of effectively alleviating chronic pain and doing so in a manner which least impacts a person's normal daily activities.

These types of conditions may potentially be treated by the stimulation of certain regions within the brain or certain nerve fibers leading to and from the brain. One such nerve is the vagus nerve, which is located in the side of the neck and acts as a highway of information for carrying messages to and from the brain. The vagus nerve is connected to many areas of the brain which are involved in detecting chronic pain as well as areas which are instrumental in producing seizures and spasms, such as those symptoms associated with Parkinson's disease and epilepsy.

Therapeutic treatment of internal organs and regions within the body have sometimes involved electrical or hyperthermic treatments. For instance, treatment modalities have included delivering energy, usually in the form of RF or electrical energy, for the heating of, e.g., malignant tumors. But many of these treatments are performed through invasive surgery (laparoscopic or otherwise) that may require repeated procedures to achieve the desired effect.

Methods used in treating epilepsy include vagal nerve stimulation, where the vagal nerve is electrically stimulated to disrupt abnormal brain activity. This may include implanting an electrical stimulation device within a patient that is electrically connected to a portion of the vagal nerve. However, this method of treatment is limited to epilepsy and may not be effective in the treatment of other types of disorders.

### SUMMARY OF THE INVENTION

Various devices and methods for cooling selected regions within a body are described herein. For example, an implantable cooling system used to cool nerve bodies such as the vagus nerve. Cooling these certain regions within the body from about 37° C. down to about 15° C. can aid in diminishing or masking impulses to control seizures, chronic pain, or otherwise treat disease.

Such an implantable cooling system may comprise an implantable unit that may contain a pumping mechanism and/or various control electronics. It may also include a heat exchanger connected to a heat sink contained within the body or that may be a part of the body. Such a heat sink can include tubular body organs through which heat may be effectively dissipated, such as the superior vena cava (SVC) or the inferior vena cava (IVC) because of the relatively high blood flow rate therein.

Additionally, the cooling system may comprise a variety of cooling devices, but it can be an electrically controllable thermoelectric module that may essentially function as a heat pump. Such modules are typically known as Peltier junctions and are generally comprised of layers of at least two dissimilar metals. When an electric current is applied to such a module, heat is moved from one side of the module to the other, thereby creating a “cool” side due to the Peltier Effect and a converse “hot” side due to the Seebeck Effect. Despite the reversible polarity of the current and the resulting reversible heating and cooling effect, the side contacting the nerve body below is called the cooled region, and conversely the side which is heated is called the heated region for simplicity. It is the cooled region which may be placed into intimate contact with the various regions within the body to effect the cooling of the appropriate tissue.

The heated region may be placed in thermal contact with a heat exchanging chamber filled with a liquid heat transfer medium. The liquid heat transfer medium can be a fluid which has a high specific heat capacity and is also biocompatible. Such fluids may include chilled saline, fluorinated hydrocarbon, such as C₆F₁₄ (e.g., Fluorinert™, by 3M, St. Paul, Minn.), liquid chlorodifluoromethane, water, air, etc., among others. Additionally, surfactants or other wetting agents can be added to the fluid to improve efficiency of the heat transfer between the fluid and the heat exchanger. As the heat transfer medium absorbs the heat from the heated region, the medium may be urged by a pump to pass through a controllable outlet and through a feedline to the second heat exchanger, where the absorbed heat may be discharged to the SVC, IVC, or other body organ.

The cooling device or unit may comprise a variety of configurations. One configuration is a semi-circular configuration where the cooled region is circumferentially surrounded by the heated region. Each of the cooled and heated regions may define an opening through which the vagus nerve or other nerve body to be cooled may pass through to enable the junction to fixedly attach about the nerve. To effect heat transfer between the junction and the nerve body, biocompatible adhesives having a sufficient thermal conductivity, i.e., does not impede the heat transfer, may be used as a thermal interface between the two. Other configurations may include clamping members which may be urged open to allow for placement onto the nerve body, and helical variations which may be unraveled temporarily by an external force to allow for placement around the nerve body. Upon releasing the external force, the device may reconfigure itself to reform its helical configuration and wrap around the nerve body.

The pump may be a conventional implantable pump with an integrated power supply and/or control electronics. Alternatively, the power supply to actuate the pump and cooling unit may be supplied by an implantable transcutaneous charger. Such a charger may have its power supply recharged by an external charging unit which may be placed over the skin in proximity of the charger. Other types of pumps may be subcutaneously implanted and externally actuated and driven. Such pumps may have a diaphragm attached to an actuator, which may comprise a permanent magnet, in the pumping chamber. The diaphragm and pump may then be actuated by an external alternating electromagnet placed over the skin. Other types of pumps may also include rotational pumps that are subcutaneously implanted and also externally actuated.

The heat exchangers which may be in contact with the tubular body organs may be configured in a variety of ways. Functionally, a heat exchanger which maximizes the contact surface area between the exchanger and the body organ is desirable. Also, the exchanger can be configured to hold onto the tubular body organ without damaging the tissue in any way. Such configurations may include a cuff-type design in which a heat exchanger element may be configured into a looped or alternating manner to increase the surface area traversed by the fluid medium as it travels through the cuff. Alternatively, the cuff may define a single continuous heat exchange chamber through which the fluid medium may fill before exiting through an outlet line and back to the cooling unit. The heat exchanger cuff, as well as the other portions of the cooling system, can be made from a biocompatible metal or alloy, e.g., stainless steel, nickel titanium, etc.

A combination implantable pump and heat transfer device may also be used in the cooling system. This variation may comprise an injectable pump having a dual-chambered body, e.g., an aspiration and an irrigation chamber. The chambers may be accessible through the patient's skin by insertion of a multi-lumened catheter having at least one lumen in fluid connection with the aspiration chamber and at least one lumen in fluid connection with the irrigation chamber. When the cooling system is to be actuated, the catheter may be inserted through the skin and the heated or charged fluid medium may be drawn into the aspiration chamber and up into the lumen while cooled fluid medium may be pumped or urged into the irrigation chamber and into the system via the other lumen.

The fluid lines transporting the fluid medium through the cooling system may comprise separate lines for the heated or charged fluid and the cooled fluid medium. Alternatively, a single multi-lumened line may define separate fluid lines therein as well as additional access lumens to carry the electrical, control, and/or power lines to minimize the number of separate lines running between units of the cooling system. The lines may be made from a variety of conventionally extrudable or formable materials, e.g., silicone, polyethylene (PE), fluoroplastics such as polytetrafluoroethylene (PTFE), fluorinated ethylene polymer (FEP), perfluoroalkoxy (PFA), and thermoplastic polymers, such as polyurethane (PU), etc.

Moreover, to prevent any kinking or undesirable bending of the fluid lines when implanted within a body, the lines may be reinforced by wrapping, braiding, or surrounding them with various metals or alloys, as is well known in the catheter arts. Examples of such metals and alloys include stainless steels, nickel titanium (Nitinol) alloys having superelastic alloy characteristics, and other superelastic alloys. Additionally, the fluid lines may also be surrounded by insulative materials to minimize any undesirable heat transfer from or to the fluid medium contained therein.

### BRIEF DESCRIPTION OF THE DRAWINGS

FIG. 1 shows a schematic of an embodiment of the cooling device with a pump.

FIG. 2 shows a schematic of an embodiment of the cooling device with a pump having electronics.

FIG. 3A shows a top view of an embodiment of the cooling element having a Peltier junction.

FIG. 3B shows an isometric view of an embodiment of the cooling element from FIG. 3A.

FIG. 4 shows a clamp variation of the cooling element.

FIG. 5 shows a helical variation of the cooling element.

FIG. 6 shows a segmented variation of the cooling element.

FIG. 7 shows a flexible sheet variation of the cooling element in a straight configuration.

FIG. 8 shows the variation of the cooling element of FIG. 7 in a partially curled, curved, or wrapped configuration.

FIG. 9 shows an externally controllable variation on the pump.

FIG. 10 shows an internally rotating variation on the pump.

FIG. 11 shows a transparent isometric view of one variation on the heat exchanger cuff.

FIG. 12 shows a transparent isometric view of another variation on the heat exchanger cuff.

FIG. 13 shows an isometric view of a device for injecting and aspirating coolant through the skin.

FIG. 14A shows a representative schematic of a variation on the cooling device having a single multi-lumened coolant tube.

FIG. 14B shows cross-section A-A from FIG. 14A of the variation on the multi-lumened coolant tube.

FIG. 15 shows an embodiment of the coolant tube wrapped with a metallic ribbon.

FIG. 16 shows an embodiment of the coolant tube braided with a metallic ribbon.

FIG. 17 shows an embodiment of the coolant tube covered with an insulative material.

FIG. 18 shows an embodiment of the cooling element.

FIG. 19 shows cross-section B-B of FIG. 18.

FIG. 20 shows a perspective view of cross-section C-C of FIG. 19.

FIG. 21 shows an embodiment of the cooling element.

FIG. 22 shows cross-section D-D of FIG. 21.

FIG. 23 shows a perspective view of cross-section E-E of FIG. 22.

FIG. 24 shows an embodiment of the cooling element.

FIG. 25 shows a perspective view of cross-section F-F of FIG. 24.

FIGS. 26 through 28 show an embodiment of a method of deploying the cooling element around a nerve.

FIG. 29 shows a variation of the cooling device implanted within a body and attached to the superior vena cava and a vagal nerve.

FIG. 30 shows a variation of the cooling device implanted within a body and attached to the superior vena cava and a region within the brain.

FIG. 31 shows an embodiment of using the cooling device attached to the posterior and anterior trunks of the vagus nerve.

FIG. 32 shows a partial see-through view of the leg illustrating an embodiment of using the cooling device attached to the femoral and sciatic nerves.

FIG. 33 illustrates a perspective view of a sagittal sectioning of a length of the spinal column.

FIG. 34 illustrates cross-section G-G of the spinal column.

FIG. 35 illustrates cross-section H-H of the spinal column.

FIG. 36 shows a sagittal section of vertebrae with a catheter inserted within the vertebral canal to cool a portion of the spinal column.

FIG. 37 illustrates an embodiment of a method of deploying an embodiment of the cooling element into the epidural space.

FIGS. 38, 40, 42, and 43 illustrate an embodiment of a method of deploying an embodiment of the cooling element into the epidural space.

FIG. 39 illustrates cross-section J-J of FIG. 38.

FIG. 41 illustrates cross-section K-K of FIG. 40.

FIGS. 44 and 45 illustrate various embodiments of cross-section L-L of FIG. 43.

FIG. 46 illustrates an alternate embodiment to the deployment configuration of FIG. 43.

FIG. 47 illustrates cross-section M-M of FIG. 46.

FIG. 48 illustrates a posterior view of an embodiment of the cooling element in a deployed configuration as shown in FIG. 47.

FIG. 49 shows an embodiment of using the cooling devices attached to the esophagus, pylorus and fundus.

FIG. 50 shows a cooling system utilizing a strain gauge attached or adhered to a stomach.

FIG. 51 shows a cooling system in which an intragastric sensor may be placed against a stomach serosal or mucosal surface and which transmits wirelessly to a controller.

FIGS. 52A and 52B illustrate a cooling system utilizing an esophageal activation sensor to detect esophageal distension and the various distension patterns which may be used to determine whether the cooling elements require activation, respectively.

FIG. 53 shows an example where a cooling unit may be activated by an external remote.

FIG. 54A illustrates a cooling element configured as a helical cooling element.

FIGS. 54B and 54C show top and side views of the controller and cooling unit.

FIG. 55 shows an example in which heat generated from the cooling element may be dissipated directly into the underlying tissue, e.g., the stomach, via the controller and cooling unit.

FIG. 56 shows another example in which the heat from the cooling element may be alternatively dissipated into other tissue structures, such as the bladder.

FIGS. 57A and 57B show examples of how a controller may be adhered or attached against the serosal or mucosal tissue layer of the stomach, respectively.

FIG. 58A illustrates another example of a cooling unit utilizing conductive heat transfer to dissipate generated thermal energy.

FIGS. 58B and 58C show various cross sectional areas of the thermal conduction line of the device of FIG. 58A.

FIG. 58D illustrates another example where a separate thermally conductive strap may be used to conduct heat away from the cooling element and into surrounding tissue structures.

### DETAILED DESCRIPTION OF THE INVENTION

Devices and methods for the controlling the temperature of selected regions within a body are described herein. The temperature of the selected region can be increased (i.e., heated), for example for nervous system stimulation. The temperature of the selected region can be decreased (i.e., cooled), for example for suppression of transmission of signals within the nervous system.

Only for the purposes of simplicity and clarity of the description, the devices and methods are repeatedly referred to herein as configured and used for cooling. All embodiments of the devices and methods described herein can be used for heating and cooling. For Peltier devices, reversing the polarity of current can reverse the direction of heat transfer (i.e., from heating to cooling or from cooling to heating). For devices using fluid cooled or heated by non-Peltier devices, or heater or cooler can be used to heat or cool the fluid to produce the desired result.

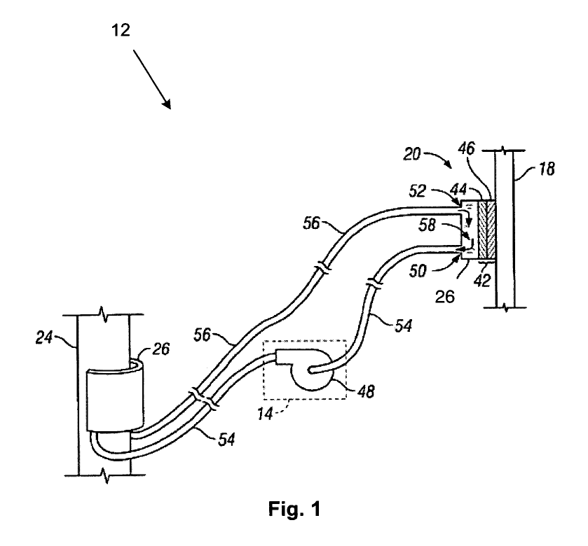

FIG. 1 shows an embodiment of a cooling system 12. The cooling system 12 can have an implantable unit 14. The implantable unit 14 can be thermally connected via coolant feedline 54 to cooling unit 20. The cooling unit 20 can be in intimate contact with a fibrous nerve body 18, such as the vagus nerve. Implantable unit 14 can be coated with any variety of biocompatible polymer and/or have a housing made from a biocompatible metal or alloy, such as stainless steel. Unit 14 can have a pump, electronics for controlling the system 12, a power supply, and/or any combinations thereof. The unit 14 may be thermally connected via coolant feedline 54 to heat exchanger 26. Heat exchanger 26 can be in thermal or heat conductive contact with a tubular body organ through which heat may be effectively dissipated, such as the superior vena cava (SVC) 24, as shown in the figure, or inferior vena cava (IVC) 30 or other large vascular members.

The cooling unit 20 may be comprised of a variety of cooling devices. The cooling unit 20 can remove heat from nerve body 18 and the surrounding region. For example, the cooling unit 20 can be a heat pump.

The cooling unit 20 can be an electrically controllable thermoelectric module. The electrically controllable thermoelectric module can be a Peltier junction 42. The Peltier junction 42 can have a sandwich of at least two carefully chose dissimilar metals, alloys, or intermetallic compounds. When an electric current is applied to the Peltier junction 42, heat can be moved from one side of the junction to the other, creating a “cool” side due to the Peltier Effect and a “hot” side due to the Seebeck Effect. If the polarity of the current is reversed, the opposite effect occurs in the respective sides of the junction. The side undergoing the Peltier Effect (or “cool” side) may be made, for instance, from bismuth telluride (Bi₂Te₃) and the side undergoing the Seebeck Effect (or “hot” side) may be made from lead telluride (PbTe), silicon-germanium (SiGe), or also Bi₂Te₃. To ensure biocompatibility when implanted, the metals or alloys of cooling unit 20 can be made of biocompatible materials. The thermoelectric module can have a lack of moving parts, lack of vibration and noise, small sizes and configurable shapes, a long module life and precise temperature control, and combinations thereof. Despite the reversible polarity of the current and the resulting reversible heating and cooling effect, the side of the cooling unit or device contacting the nerve body is called herein the cooled region, and conversely, the side which is heated is called the heated region for simplicity and clarity.

The Peltier junction 42 can have the cooled region 46 placed in close contact against or around nerve body 18. As cooled region 46 is cooled, heated region 44 conversely heats up. Heat exchanger 26 can have a chamber filled with a liquid heat transfer medium 58. The heat exchanger 26 can be in direct and/or thermal contact with heated region 44. The liquid heat transfer medium 58 can be a fluid that can have a high specific heat capacity. The liquid heat transfer medium 58 can be biocompatible. The liquid heat transfer medium 58 can be chilled saline, fluorinated hydrocarbon (Fluorinert™), liquid chlorodifluoromethane, water, air, or combinations thereof. A pump 48 can be in the implantable unit 14. The pump 48 can be fluidly connected via the coolant feedline 54 to the cooling unit 20. As heat transfer medium 58 absorbs the heat from heated region 44, medium 58 can be urged to pass through a controllable outlet 50 through the feedline 54 and through the implantable unit 14 by the pump 48. From the pump 48, the heated medium 58 can travel through the feedline 54 to the heat exchanger 26, where the absorbed heat may be transferred to the body organ (e.g., SVC 24), against or near the body surface (not shown) or external to the body (not shown).

The heat exchanger 26 can be in intimate contact with a hollow body organ which is able to act as a heat sink and absorb the heat which may be discharged from the medium 58 as it flows through the heat exchanger 26. The heat exchanger 26 can be made from a biocompatible metal or alloy (e.g., stainless steel) which has an adequate thermal conductivity value such that heat from medium 58 may be effectively transferred through exchanger 26 and external to the body or into the hollow body organ to which the exchanger 26 is contacting. Hollow body organs which generally have a high blood flow rate and which may functionally act as heat sinks include SVC 24, as shown in the figure. Heat exchanger 26 can be configured to intimately covers a portion of SVC 24 substantially around the circumference of the SVC 24, i.e., around at least a majority of the circumference of SVC 24. The heat exchanger 26 can be formed in a cuff-shaped configuration. The heat exchanger 26 can securely clamp around the hollow body organ to prevent excessive movement or dislodgment. A biocompatible adhesive which has an effective thermal conductivity value can be filled between heat exchanger 26 and SVC 24 to aid in optimizing heat transfer and attachment to SVC 24.

The heat exchanger 26 can be placed at a location just beneath or close to the skin. During the heat exchanging process, the fluid medium 58 flowing through implanted exchanger 26 can be cooled by regular conduction and/or by supplemental external methods, such as placing a cooling device like a package of ice over the skin adjacent to the implanted exchanger 26. The fluid medium 58 can then flow through coolant return line 56 to cooling unit 20, for example once the fluid medium 58 has had the heat energy sufficiently discharged. In the cooling unit 20 the fluid medium 58 can pass through an optionally controllable inlet 52 into heat exchanger 40 to begin the process again.

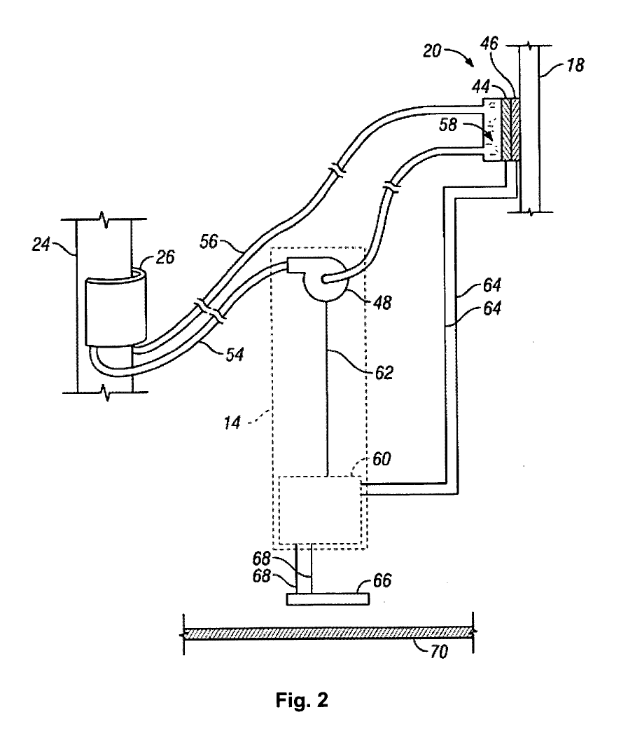

FIG. 2 shows a representative schematic of another variation on the cooling device having a pump 48 and corresponding electronics in implantable unit 14. Unit 14 can be configured to have a power supply and/or control electronics 60 enclosed within the same housing as the unit 14. The control electronics 60 can be electrically connected via control line 62 to pump 48 to supply power and/or control the pump 48. The control electronics 60 are connected via control lines 64 to cooling unit 20 to each of heated and cooled regions 44, 46, respectively. The control electronics 60 can contain a power supply and/or can be connected via electrical connection lines 68 to a charger 66 which can be subcutaneously implanted. The charger 66 can be placed below skin 70 such that an external charging unit (not shown) can be placed over skin 70 in the proximity of charger 66 such that unit 60 may be electrically (e.g., inductively or otherwise magnetically) charged thereby.

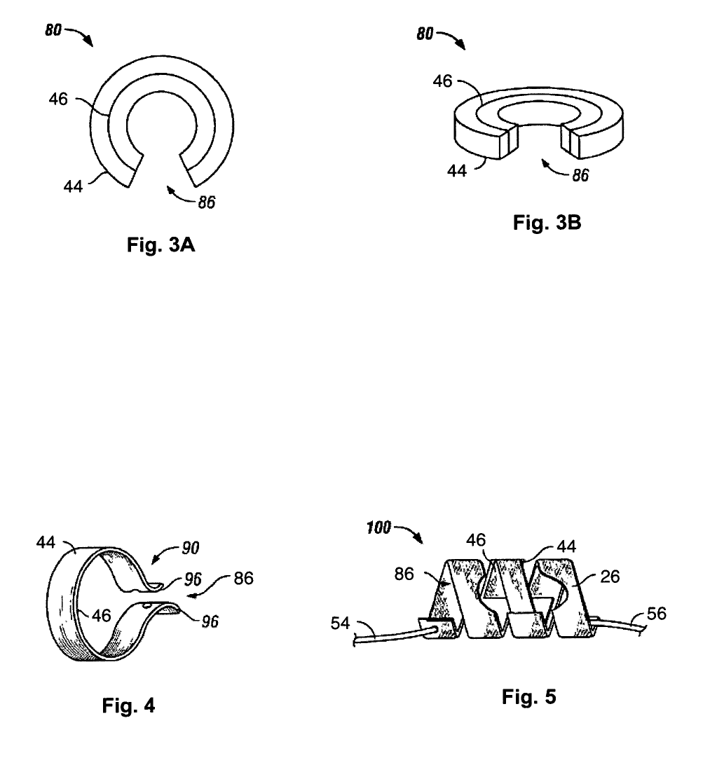

FIG. 3A shows the cooling unit 20 that can have a circular junction 80 where the cooling unit 20 can be shaped in a semi-circular configuration where cooled region 46 is circumferentially surrounded by heated region 44. Cooled and heated regions 46 and 44 can define an opening 86 through which the nerve 18 can pass to enable junction 80 to fixedly attach to the nerve 18. Junction 80 can be configured to be a size small enough to intimately attach to a nerve body 18, yet large enough to effect sufficient heat transfer to cool the nerve body 18 temperature down by several degrees. A biocompatible adhesive that can have a sufficient thermal conductivity, i.e., at least does not substantially impede the heat transfer or function as an insulator, may be used as a thermal interface between cooled region 46 and the nerve body 18.

FIG. 3B shows an isometric view of junction 80 where opening 86 and the configuration of both regions 46, 44 can be seen. The heat exchanger may be similarly configured to overlay junction 80 circumferentially or otherwise, but has been omitted for clarity.

FIG. 4 shows that a clamp variation 90 of the cooling element can have a cooled region 46 positioned interiorly of heated region 44 (e.g., in a manner similar to the variation shown in FIGS. 3A and 3B). The clamped junction 90 variation can have clamping members or arms 96 biased towards one another such that the tendency of clamping members 96 is to close opening 86. In use, members 96 can be urged open to allow for placement onto the nerve body 18. Once appropriately positioned, members 96 can be released to allow the junction 90 to securely fasten onto the nerve body 18.

FIG. 5 shows a side view of a helical variation 100 of the cooling element. In this variation, cooled region 46 may be seen as being located interiorly of heated region 44. Surrounding heated region 44 is heat exchanger 26, shown partially removed to illustrate cooled and heated regions 46, 44, respectively. Leading to a first end of device 100 is feedline 54 through which fluid medium 58 may enter heat exchanger 26. At the opposing second end of device 100 is return line 56 through which fluid medium 58 may exit heat exchanger 26 once having absorbed the heat. Heat exchanger 26 may be helically formed as shown, or it may simply be configured as an overlaying layer through which the fluid medium 58 may flow therethrough. The device 100 can be flexible enough such that it may be unraveled temporarily by an external force to allow for placement around the nerve body 18. Upon releasing the external force, the device 100 can reconfigure itself to reform opening 86 and wrap around the nerve body 18 and reform the helical configuration. The helical shape may allow for efficient heat transfer from the nerve body 18 to the device 100 given the surface contact area between the two bodies.

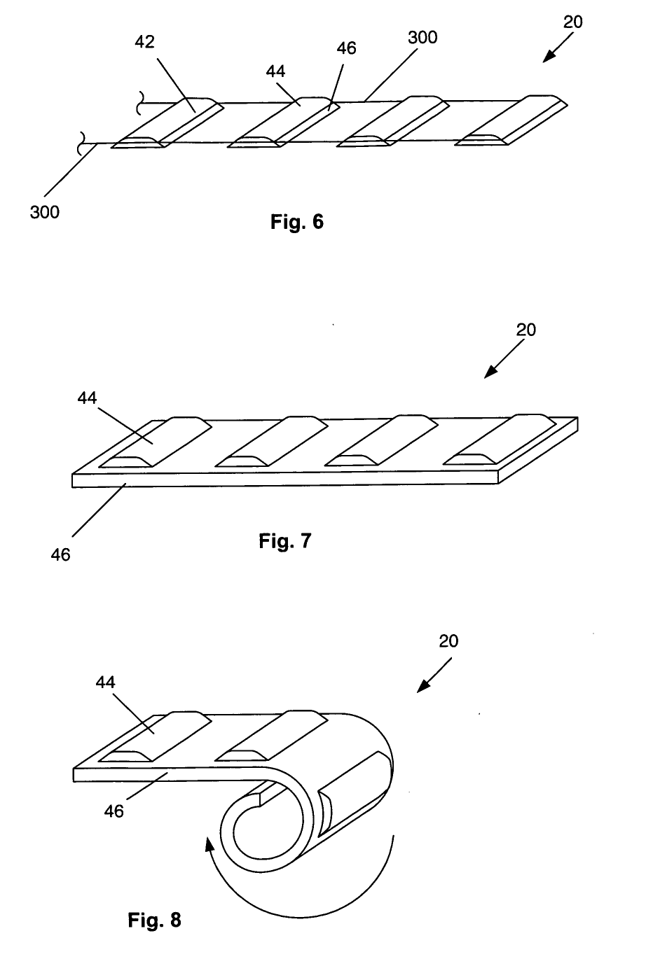

FIG. 6 illustrates that the cooling unit 20 can have numerous and/or segmented cooling cells, such as Peltier cells 42. The Peltier junctions 42 can be attached by connecting leads 300. The connection leads 300 can be configured to provide electrical current. For example, the connecting leads 300 can have electrically conductive wires that can be in electrical communication with the heated 44 and/or cooled 46 regions of the Peltier cells 42.

The connecting leads 300 can be flexible. The connecting leads 300 can be deformable. The connecting leads 300 can be strong enough to maintain a configuration after being deformably bent. The connecting leads 300 can be resilient.

The connecting leads 300 can be configured to have a hollow connecting lead channel (not shown), for example for the flow of liquid heat transfer medium. The Peltier cells 42 can have a hollow Peltier cell channel (not shown) in the heated 44 and/or cooled 46 regions, for example in fluid communication with the hollow connecting lead channel, for the flow of liquid heat transfer medium 58.

FIG. 7 illustrates that numerous and/or segmented heated regions 44 can be attached and/or integral with a cooled region 46. The heated regions 44 can be on a single side of the cooled region 46. The heated regions 44 can be spaced evenly along a longitudinal axis of the cooled region 46. The heated regions 44 can cover about 50% or more than about 50% of the surface area of the side of the cooled region 46 onto which the heated regions 44 are attached and/or integrated.

The cooled region 46 and/or heated regions 44 can be flexible. The cooled region 46 and/or heated regions 44 can be deformable. The cooled region 46 and/or heated regions 44 can be resilient. The cooled region 46 and/or heated regions 44 can be made from a shape memory alloy, such as Nitinol. FIG. 7 illustrates that the cooled region 46 can be made from a shape memory alloy that is biased to a flat configuration or biased to a curved configuration and forced into a flat configuration. FIG. 8 illustrates that the cooled region 46 can be biased in a curved configuration, but forced to a partially flat (i.e., on the left side when viewing the figure) configuration and released to a partially curved, relaxed configuration (i.e., on the right side when viewing the figure). The cooled region 46 can actively curve when-released from a sufficient resistance force and/or heated, as shown by arrow.

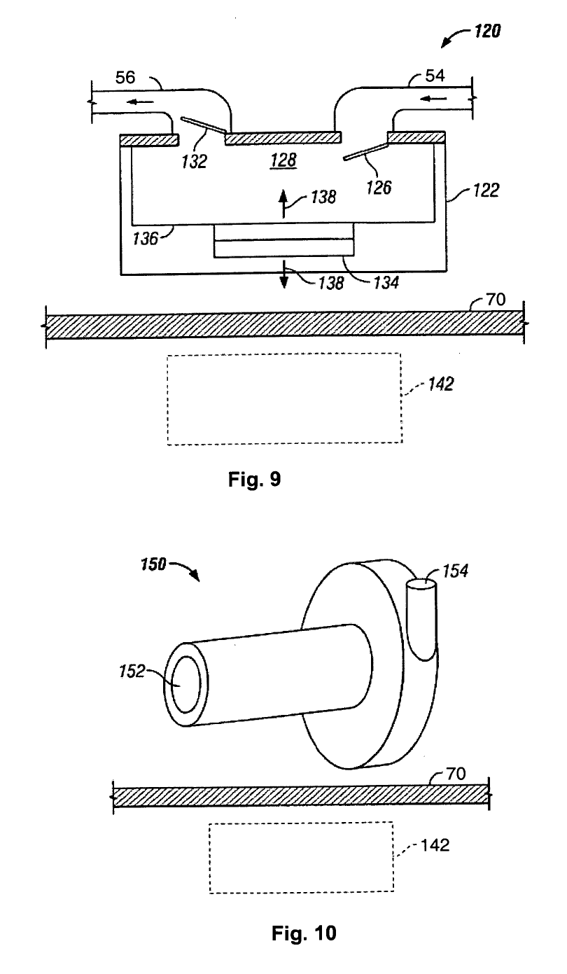

The pump 48 can urge the fluid heat transfer medium 58 through the system from cooling unit 20 to heat exchanger 26. The pump 48 can be powered by implanted power supplies or power supplied external to the patient's body. An implanted power supply may be transcutaneously charged periodically. As shown in FIG. 9, the pump can be an externally-driven pumping mechanism 120. A cross-sectioned view of externally controllable and externally driven pump body 122 may be seen attached to fluid inlet line 54. The fluid medium 58 may be transported via inlet line 54 through inlet valve 126 and into pumping chamber 128. From pumping chamber 128, the fluid medium 58 may be forced out into outlet line 56 through outlet valve 132. The inlet and outlet valves 126, 132 can be actively controllable. The inlet and outlet valves 126, 132 can be conventional valves configured to maintain unidirectional flow of the fluid medium.

The pump variation 120 does not require an implanted power supply. The pump variation 120 can be implanted subcutaneously near the skin 70. When pumping is to be actuated, an external alternating electromagnet 142 can be placed over skin 70 to activate actuator 134, which may comprise a permanent magnet. The actuator 134 can be located next to pumping chamber 128 within the pump 120. Actuator 134 can be attached to diaphragm 136. When electromagnet 142 activates pump 120, actuator 134 may oscillate in the direction of arrows 138 at a controllable frequency to drive diaphragm 136. The diaphragm 136, for example when oscillating, can urge the fluid medium 58 into and out of chamber 128. Alternating electromagnet 142 can be an externally held electromagnet. The electromagnet 142 can be strapped into place when in use and removable when not in use.

FIG. 10 shows an alternative variation of the pump as a rotational pump 150. Rotational pump 150 may be implantable. Rotational pump 150 can comprise a rotary pumping mechanism. The fluid medium 58 can enter through inlet 152 and be urged out through outlet 154. Rotational pump 150 can be subcutaneously implanted beneath skin 70. Access to the pump 150 via an externally positioned alternating electromagnet 142 may be possible for actuating the pumping mechanism. Examples of rotational pump 150 may be seen in U.S. Pat. No. 5,840,070 to Wampler, which is incorporated herein by reference in its entirety.

The heat exchangers which may be in contact with the tubular body organs may be configured in a variety of ways. The heat exchanger can maximize the contact surface area between the exchanger and the body organ. The exchanger can be configured to hold onto the tubular body organ without damaging the tissue.

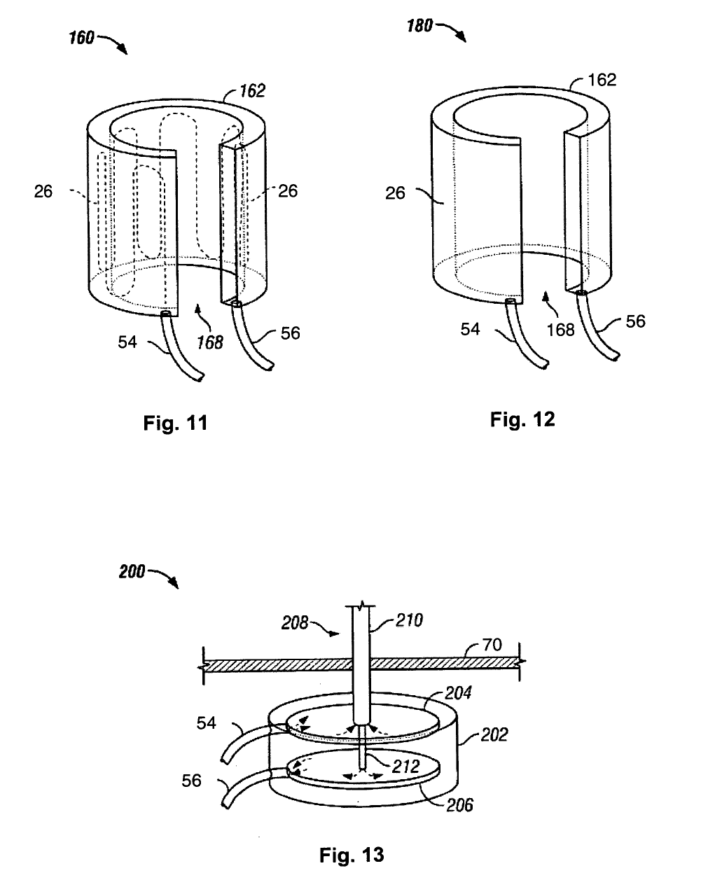

FIGS. 11 and 12 show transparent isometric views of two alternative heat exchanger cuff designs. Heat exchanger variation 160 shows cuff 162 having an inlet line 54, which may transport the heated (charged) fluid medium from the pump. The heat exchanger element 26 in this variation is configured into a looped or alternating manner to increase the surface area traversed by the fluid as it travels through cuff 162. Contact area 168 may thus optimize the heat transferred from the fluid medium 58 into the tubular body organ through contact area 168. After the fluid medium 58 has traversed through exchange element 26, the fluid medium can be channeled out of cuff 162 through outlet line 56 to be returned toward the nerve body 18 (e.g., to absorb more heat). Cuff 162 can be made from a biocompatible metal or alloy, e.g., stainless steel, nickel titanium, gold, platinum, which has a thermal conductivity value sufficient for transferring the heat from the fluid medium and through contact area 168.

FIG. 12 shows the heat exchanger 180 that can have cuff 162 attached to inlet and outlet lines 54, 56, and a contact area 168 similar to variation 160 shown in FIG. 11. The heat exchanger 180 can have a single continuous heat exchange chamber 26 through which the fluid medium may fill before exiting through outlet line 56.

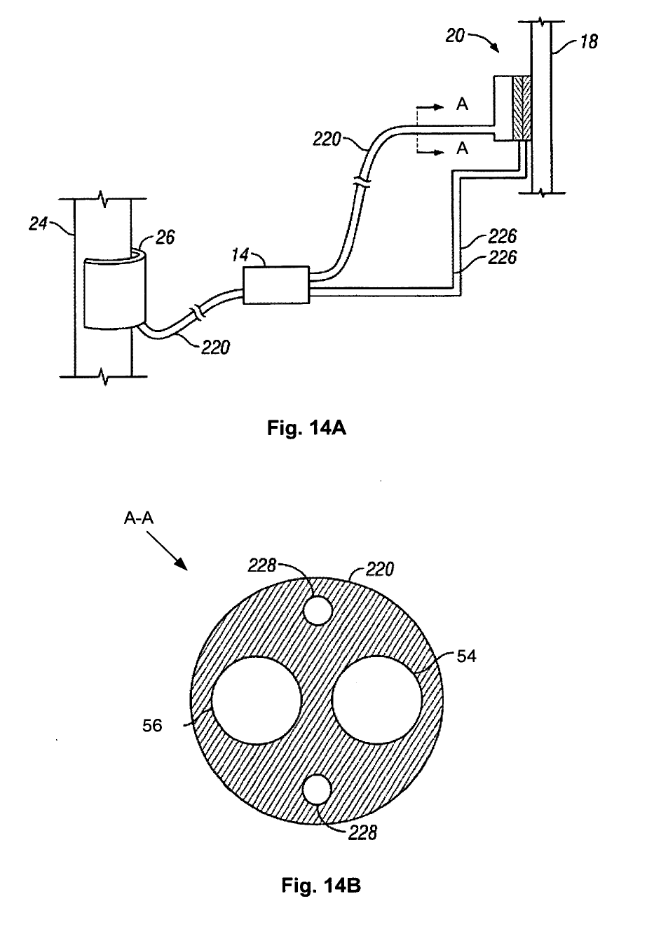

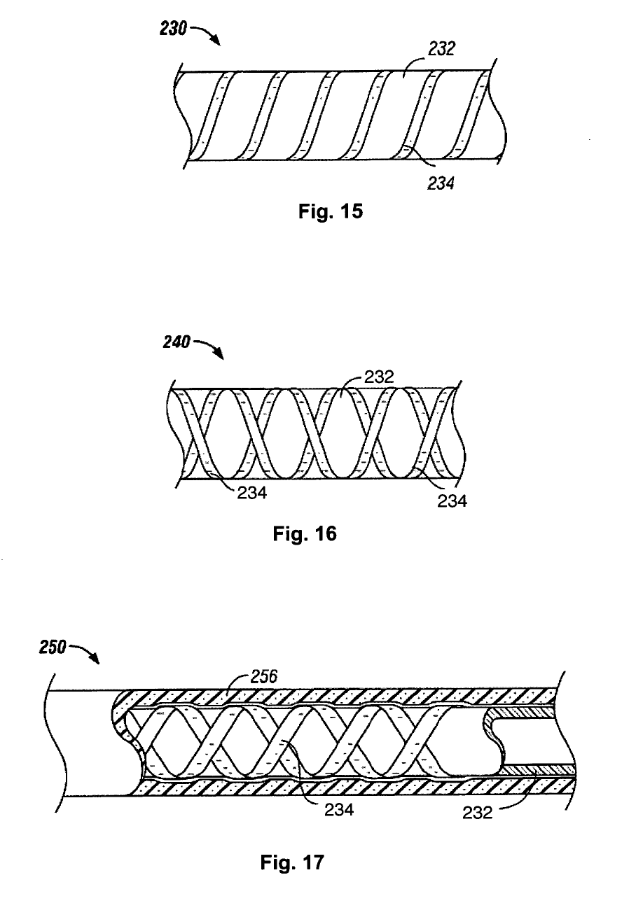

A combination pump and heat transfer device can be used in the cooling system, such as one shown in injectable pump 200 of FIG. 13. Injectable pump 200 is shown in this variation as a dual-chambered body 202 which may have an aspiration chamber 204 and an irrigation chamber 206 contained therein. Pump 200 can be implanted in the patient's body subcutaneously, for example, such that access to body 202 is possible by inserting a multi-lumened catheter 208 through skin 70. Catheter 208 can have an outer tubular structure 210 and an inner tubular structure 212. The outer tubular structure 210 and an inner tubular structure 212 can have access lumens therethrough. When the cooling system is actuated, catheter 210 can be inserted through skin 70 where the heated or charged fluid medium may be drawn into aspiration chamber 204 via inlet line 54 and up into outer tubing 210. Cooled fluid medium can be pumped or urged into irrigation chamber 206 via inner tubing 212 and into the system via outlet line 56. A positive or negative pressure pump may be fluidly connected to catheter 208 externally of the patient body to urge the fluid medium through the system. A syringe can be used to urge the fluid through the system. Multi-lumened catheter 208 can be insertable through skin 70. The multi-lumened catheter 208 can be permanently attachable to body 202 such that a proximal end of catheter 208 is maintained outside the patient's body and readily accessible when cooled fluid medium 58 needs to be circulated through the system.

The lines for transporting the fluid medium 58 between heat sink and heat exchanger may be contained in a single multi-lumened line. As shown in FIG. 14A, a single multi-lumened line 220 may be used to fluidly connect heat exchanger 26 with cooling unit 20 through implantable unit 14. The electrical control lines 226 connecting implantable unit 14 with cooling unit 20 may also be contained within multi-lumened line 220.

FIG. 14B shows multi-lumened line 220 may define coolant line 54 and return line 56 through which the fluid medium 58 may flow in opposing directions. To minimize the heat transferred between the two lines 54, 56 during flow, these lines can be insulated, for example, to attenuate any heat transfer from occurring therebetween. Multi-lumened line 220 may define a number of access lumens 228 in which electrical, control, and/or power lines, e.g., control lines 226, may be contained within to minimize the number of separate lines running between units of the cooling system.

The lines may be reinforced by wrapping or surrounding them with various metals or alloys, as is well known in the catheter arts, for example to reduce kinking. Examples of such metals and alloys include stainless steels, nickel titanium (nitinol) alloys having superelastic alloy characteristics, and other superelastic alloys.

FIG. 15 shows a representative side view of wrapped fluid line 230. Fluid line 232 may be wrapped in a helical manner along at least a majority of the length of the fluid line 232 by ribbon 234. Although a ribbon is shown as being used, a wire may also be used. FIG. 16 shows another variation in braided fluid line 240. In this variation, fluid line 232 may be wrapped in a braided manner by ribbon 234; wire may also be used to wrap line 232, or a combination of ribbon and wire may also be used. A further variation of a fluid line may be seen in FIG. 17, where insulated fluid line 250 may optionally be insulated with an exteriorly disposed tubular member 256. Although insulated fluid line 250 is shown with braided ribbon 234 disposed between fluid line 232 and insulation 256, the braided ribbon 234 may be disposed exteriorly of insulation 256, formed integrally within the walls of insulation 256, or even integrally within the walls of fluid line 232. The fluid lines may be made from a variety of conventionally extrudable or formable materials, e.g., silicone, polyethylene (PE), fluoroplastics such as polytetrafluoroethylene (PTFE), fluorinated ethylene polymer (FEP), perfluoroalkoxy (PFA), thermoplastic polymers, such as polyurethane (PU), and combinations thereof.

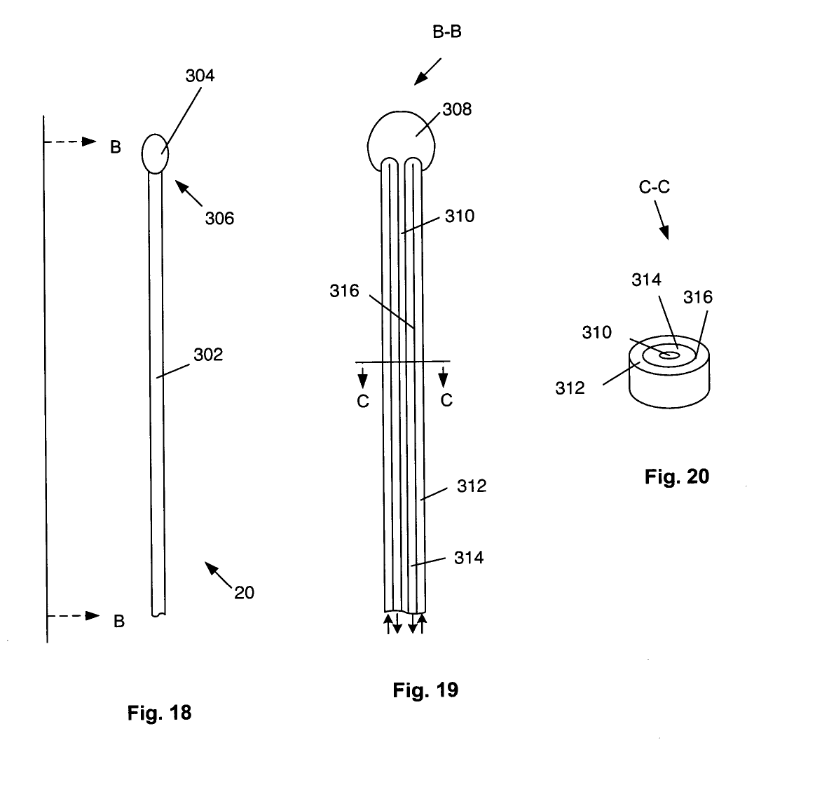

FIGS. 18 through 20 illustrate that the cooling element 20 can be a fluid conduit. In use, the fluid medium 58 can be directed through the cooling element 20. The cooling element 20 can have any or all of the characteristics of the fluid line. The cooling element 20 can have a catheter body 302 and an anchoring mechanism, such as a tip balloon 304. The anchoring mechanism can be atraumatic. The anchoring mechanism can have hooks, barbs, spikes, brads, or combinations thereof. The anchoring mechanism can have a textured surface. The catheter body 302 can be flexible and/or rigid (e.g., in alternating flexible and rigid lengths). The catheter body 302 can be deformable and/or resilient (e.g., in alternating deformable and/or resilient lengths). The distal end (e.g., near the tip balloon 304) of the catheter body 302 can have a catheter neck 306. The catheter neck 306 can be more flexible than the remainder of the adjacent region of the catheter (e.g., made from a different material, made with thinner walls).

The catheter body 302 can have a catheter body diameter from about 18 gauge needle diameter to about a 12 gauge needle diameter, for example about a 16 gauge needle diameter. The catheter body 302 can have also have a reinforcement. For example, the catheter body 302 can be surrounded by a spiral reinforcing wire (not shown) such as a coil, or a braid or weave.

The tip balloon 304 and/or the catheter body 302 can made from a resiliently expandable material. The tip balloon 304 and/or the catheter body 302 can have a reinforcing mesh (e.g., metal such as Nitinol, high strength fiber such as carbon fiber or Kevlar® from E. I. du Pont de Nemours and Company) woven into the expandable material.

The tip balloon 304 can have a hollow tip balloon cavity 308. The catheter body 302 can have a tip balloon channel 310. The tip balloon channel 310 can be in fluid communication with the tip balloon cavity 308. The tip balloon channel 310 can have a conductive tip wire. The conductive tip wire can activate electrical anchoring mechanisms. An inflation fluid can be pumped into the tip balloon channel 310 to inflate the tip balloon.

The catheter body 302 can have an outer thermal fluid channel 312 and an inner thermal fluid channel 314. The outer thermal fluid channel 312 can be in fluid communication with the inner thermal fluid channel 314, for example at or near the distal end of the catheter body 302. The outer thermal fluid channel 312 can be separated from the inner fluid channel 314 by a thermal fluid channel septum 316. The thermal fluid channel septum 316 can be insulated (e.g., thicker than the other walls of the catheter body 302, and/or made from a more resistant material than the other walls of the catheter body 302). During use, the fluid medium 58 can be pumped into the outer thermal fluid channel 312 and out of the inner thermal fluid channel 314, as shown by arrows. The fluid medium 58 can be pumped through the thermal fluid channel 316 at a fluid flow rate from about 10 m/s to about 15,000 m/s, more narrowly from about 1,000 m/s to about 10,000 m/s. The flow direction can be reversed from that shown in FIG. 19.

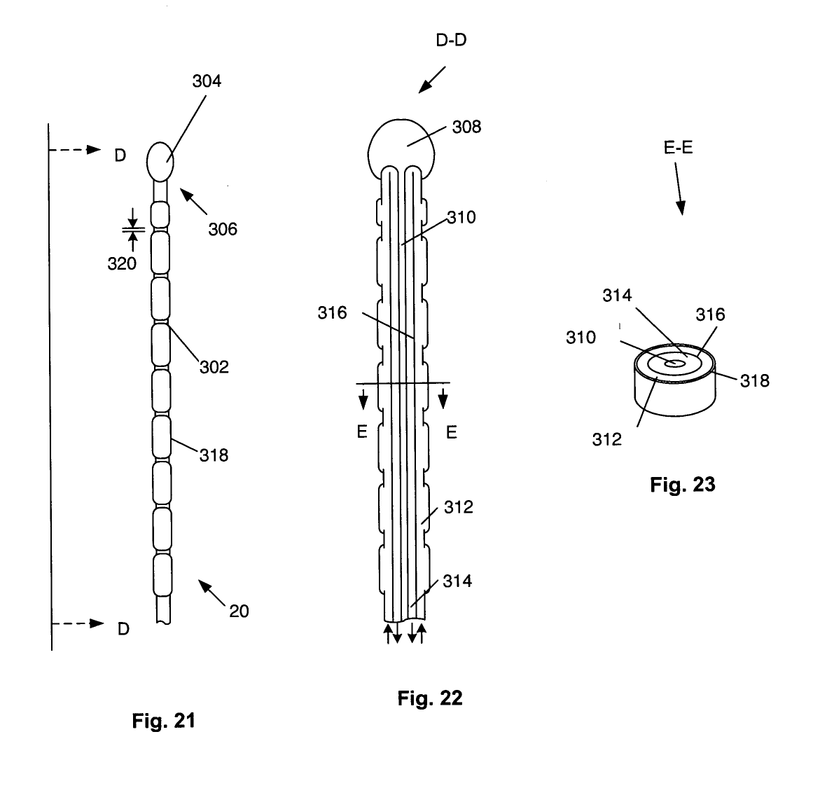

FIGS. 21 through 23 illustrate that the catheter body can have one or more body anchors, such as body balloons 318. The body anchors can have the same attributes as the anchoring mechanisms. The body balloons 318 can be separated from the other body balloons by a body balloon gap 320. Each body balloon gap 320 can be about equal to the length of either or both of the adjacent body balloons. The body balloon gap 320 can be smaller or larger than the length of the adjacent body balloons. Each body balloon 318 can completely or partially circumferentially surround the catheter body 302. The body balloons 318 can be made from the same material as the tip balloon 304. One or more or all (as shown) of the body balloons 318 can be in fluid communication with the outer thermal flow channel 312. All the body balloons 318 can have a separate balloon inflation channel (not shown). Each body balloon 318 or groups of body balloons can have individual balloon inflation channels. The body balloons can be inflated individually or as groups or as a whole.

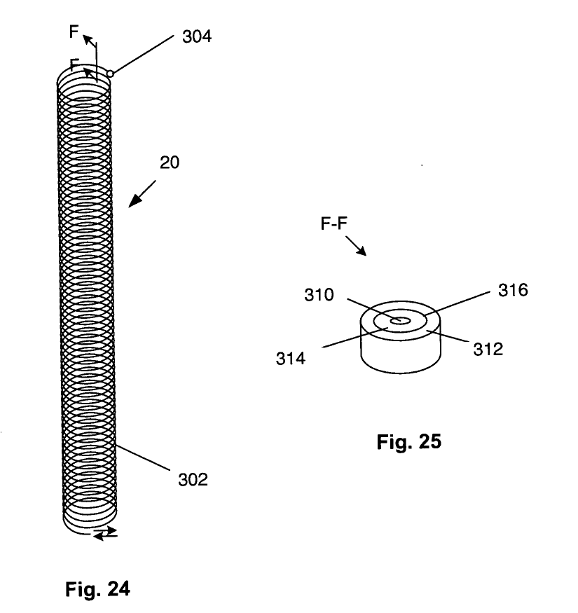

FIG. 24 illustrates that the catheter body 302 can have a coiled configuration. (The catheter body 302 is shown having a nominal thickness in FIG. 24 for simplicity and clarity.) The catheter body 302 can be made from a shape memory material (e.g., a Nitinol mesh embedded in a flexible polymer tube) that can have a coiled configuration in a relaxed state. The catheter body 302 can be forced into a straight configuration during deployment. The catheter body 302 can relax into the coiled configuration when finally deployed and/or during deployment.

Any or all elements of the cooling element and/or other devices or apparatuses described herein can be made from, for example, a single or multiple stainless steel alloys, nickel titanium alloys (e.g., Nitinol), hydrogels (e.g., the cooling element can have a hydrogel-coated Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E. I. Du Pont de Nemours and Company, Wilmington, Del.), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRA® Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the cooling element and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

The cooling element and/or elements of the cooling element and/or other devices or apparatuses described herein and/or the fabric can be coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

### Methods of Use

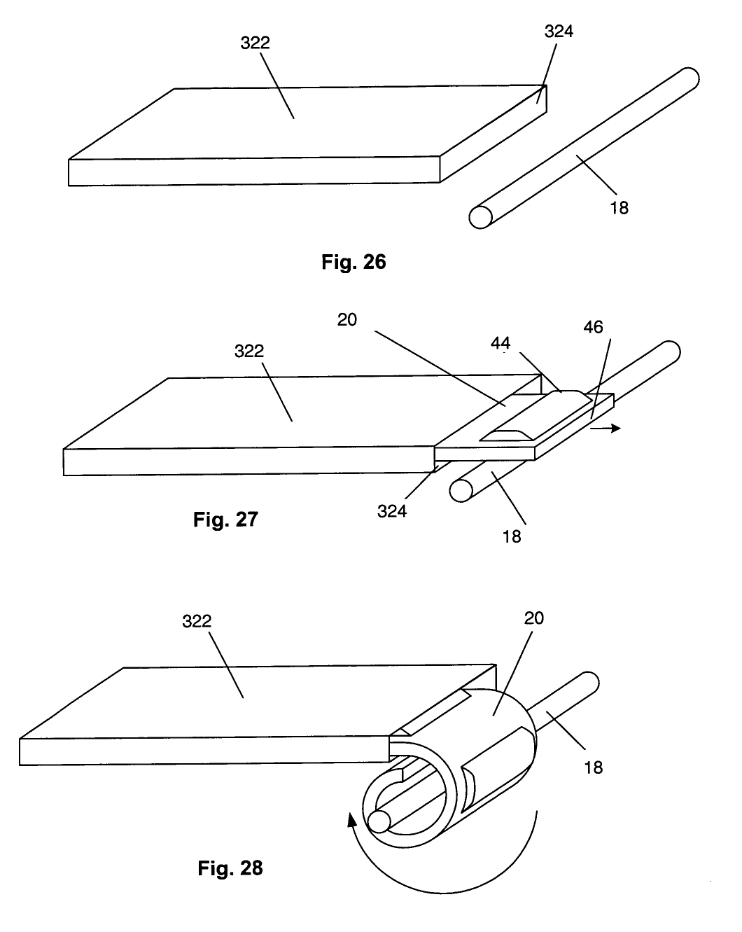

FIGS. 26 through 28 illustrate that the cooling element can be deployed around a nerve body 18. The cooling element 20 can be deployed percutaneously, for example minimally invasively or through an open procedure. As shown in FIG. 26, a deployment sheath 322 can be deployed adjacent to the nerve body 18. The distal end of the deployment sheath 322 can have a delivery port 324. The deployment sheath 322 can have a rectangular cross-section (as shown) or a round cross-section. The deployment sheath 322 can be a needle, catheter, trocar, or other access device. The deployment sheath 322 can have a deployment sheath diameter from about that of a 9 gauge needle or smaller, more narrowly that of about a 12 gauge needle or smaller, more narrowly from that of a 12 gauge needle to about that of an 18 gauge needle, for example, that of about a 16 gauge needle.

As shown by the arrow in FIG. 27, the cooling element 20 can be advanced from the delivery port 324. In the deployment sheath 322, the deployment sheath 322 can force the cooling element 20 to retain a straight configuration. The cooling element 20 can be longer than needed and length dimensions can be marked along the length of the cooling element 20. The deployment sheath 322 can have an advancer (not shown), such as a plunger. The advancer can be ratcheted. The advancer can monitor and report the length of cooling element 20 deployed at any time. The user can deploy the cooling element 20 manually or with the aid of a tool (e.g., the ratcheted advancer) to a desired length.

As shown by the arrow in FIG. 28, when advanced out of the deployment sheath 322, the cooling element 20 can relax or be forced (e.g., by a curved distal tip of the deployment sheath 322 that deforms the cooling element 20, not shown) to a curved configuration. The cooling element 20 can curl around the nerve body 18, as shown. The cooling element 20 can curl into direct contact with the nerve body 18. The cooling element 20 can compress the nerve body 18.

The nerve body 18 can be any nerve accessible by a minimally invasive, open or any other procedure. The nerve body 18 can be the alveolar, anal, anococcygeal, antebrachial, auricular, auriculotemporal, axillary, brachial, buccal, calcaneal, cardiac, caroticotympanic, carotid, celiac, cervical, chorda tympani, ciliary, cluneal, coccygeal, cochlear, cranial, crural, cutaneous, digastric, digital, dorsal, ethmoidal, femoral, fibular, ganglionic, gastric, geniohyoid, genital, genitofemoral, gingival, glossopharyngeal, gluteal, hepatic, hypogastric, hypoglossal, ilioinginal, infraorbital, infrapatellar, infratrocheal, intercostals, intercostobrachial, interosseous, intestinal, ischiatic, labial, lacrimal, laryngeal, lingual, mandibular, masseteric, maxillary, median, musculocutaneous, mylohyoid, nasal, nasociliary, nasopalatine, obturator, occipital, oculomotor, olfactory, ophthalmic, optic, palatine, palmar, palpebral, pancreatic, parotid, pectoral, pericardial, petrosal, pharyngeal, phrenic, plantar, plexus, presacral, pudendal, pyloric, quadratus plantae, radial, rectal, sacral, saphenous, scapular, sciatic, scrotal, splanchnic (e.g., greater, least, lesser, lumbar, pelvic, sacral, thoracic), stylohyoid, subcostal, sublingual, supraclavicular, supraorbital, suprascapular, supratrocheal, sural, temporal, tentorial, thoracic, thoracoabdominal, thoracodorsal, thyrohyoid, tibial, tonsillar, trigeminal, trochlear, tympanic (i.e., Jacobson), ulnar, vagus (e.g., anterior vagal trunk, auricular branch, cardiac branch, celiac branch, esophageal branch, gastric branch, hepatic branch, intestinal branch, meningeal branch, pharyngeal branch, posterior vagal trunk, pulmonary branch), vestibular, vestibulocochlear, Vidian, or zygomatic nerve(s) and branches and trunks thereof, spinal cord, dorsal roots, ventral roots, the ganglion of Impar, the nerves of Laterjet, the parts of the brain (e.g., ventricles—such as cooling cerebrospinal fluid in the ventricle—thalamus, corpus collosum), or combinations thereof. The cooling can be centralized to a specific length along the nerve 18. The cooling can be localized to a specific side of the nerve 18. For example, the cooling can be focused on the dorsal columns of the spinal cord, but not the ventral columns, or the ventral columns but not the dorsal columns.

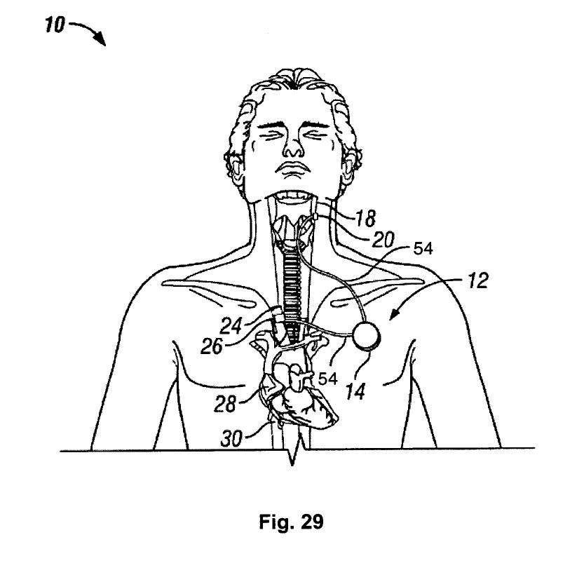

FIG. 29 shows a patient 10 having a variation of cooling-system implanted within the body. The unit 14 may be implanted in various locations within the body relative to heart 28. The unit 14 can be thermally connected via coolant feedline 54 to heat exchanger 26. Heat exchanger 26 can be in thermal or heat conductive contact with a tubular body organ through which heat may be effectively dissipated, such as the superior vena cava (SVC) 24, as shown in the figure, or inferior vena cava (IVC) 30 or other large vascular members. In operation, nerve body 18 may be cooled from normal body temperature, about 37° C., down to about 30° C., by cooling unit 20.

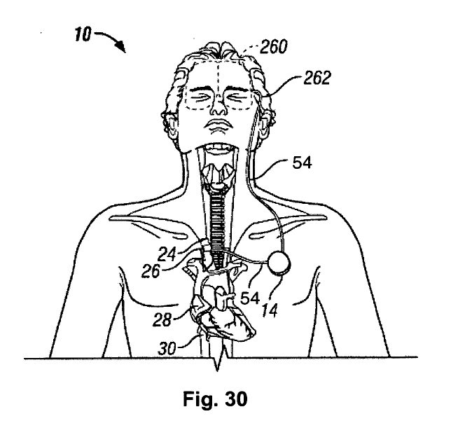

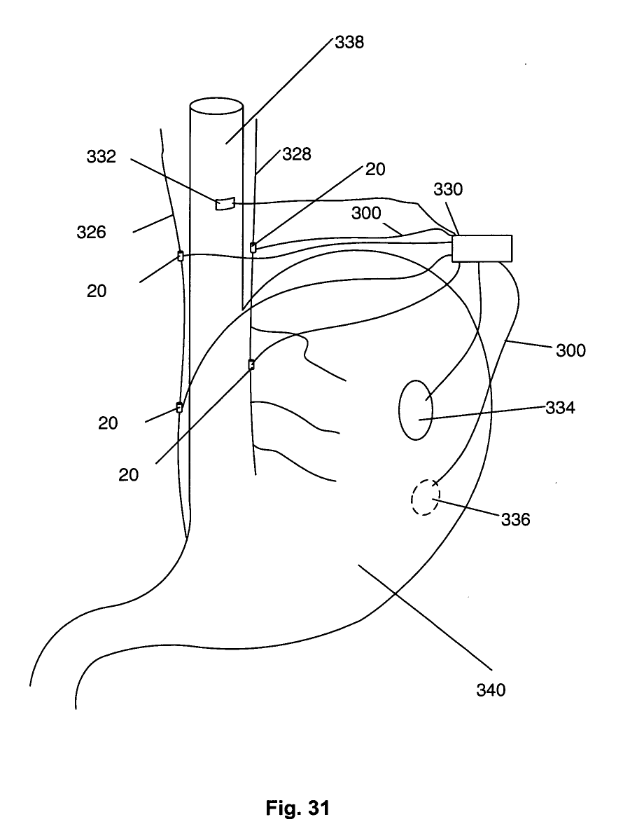

FIG. 30 shows that a probe 262 may be used for implantation within the brain 260 of a patient. The probe 262 may employ a Peltier junction 42 configured to be shaped as an elongate member for implantation within the brain 260. The elongate probe 262 may be in contact with a heat exchanger as described herein to effect heat transfer away from the brain 260. Probe 262 or variations thereof may be used for implantation within or adjacent to regions of the spinal cord to cool certain regions for the treatment of various maladies, e.g., chronic pain.

FIG. 31 illustrates that one or more cooling elements can be placed on the posterior vagus nerve trunk 326 and/or the anterior vagus nerve trunk 328. One or more cooling elements 20, and/or one or more sensors, and/or a remote control can be in power and/or data communication with a controller 330, such as the control electronics 60 described herein. The controller 330 can be implanted or outside the body. The remote control (not shown) can be outside the body. The sensors can be, for example, an esophageal activation sensor 332 and/or a stomach surface sensor 334 and/or an intragastric sensor 336. When the sensors send data to the controller 330 indicating substantive digestive activity by the esophagus 338 and/or stomach 340 and/or when activated by the remote control, the controller can activate the cooling elements 20. When the vagus nerve or branches of the vagus nerve are cooled as shown, digestive activity in the stomach can be slowed or otherwise suppressed. Slowing and/or suppressing digestive activity can suppress appetite (i.e., treating obesity).

The cooling elements 20 can be in data and/or power communication with the controller 330 via electrical, sonic, other mechanical, or radiofrequency signal via wire leads 300 and/or wirelessly (e.g., 802.11 (wireless LAN), Bluetooth, IRDA, RFID, cellular communication modem, radio such as 900 MHz RF or FM signal, microwave, ultrasound such as high-frequency ultrasound (HIFU)), or combinations thereof. The cooling elements 20 can have attached and/or integrated rechargeable electrical cells or batteries, for example as a power supply. The sensors can be connected in data and/or power communication to the controller 330 via electrical, sonic, other mechanical, or radiofrequency signal via wire leads 300 and/or wirelessly.

The remote control can be in wireless data communication with the controller 330. The remote control can be in wireless power communication with the controller 330. For example, the remote control can transcutaneously inductively charge the controller 330.

The esophageal activation sensor 332 can be partially or completely circumferentially surrounding the esophagus 338. The esophageal sensor 332 can be configured to sense myoelectric signals in esophageal muscle and/or have a strain gauge. The strain gauge can measure digestive contractions by the esophagus 338. The strain gauge can be a foil gauge. The strain gauge can be an FOS Strain Gauge by Rice Engineering & Operating Ltd. The strain gauge can be a linear optical encoder with a transmitter/receiver having a band between with small slits. Measurements from the linear optical encoder can quantify relative and absolute positions (i.e., that can be used to measure strain). The band can incorporate the measurement slits or light and/or dark marks, for example as in some optical encoders.

The stomach surface sensor 334 can be sutured or otherwise anchored to the surface of the stomach 340. The intragastric sensor 336 can be attached to the muscularis of the stomach 340 or serosa. The stomach surface sensor 334 and intragastric sensor 336 can be configured to sense myoelectric signals in stomach muscle and/or have a strain gauge. The strain gauge can measure digestive contractions by the stomach 340.

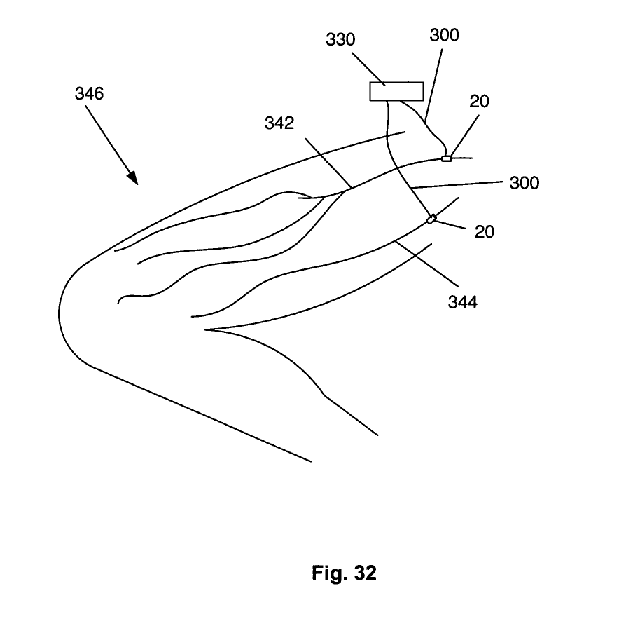

FIG. 32 illustrates that the cooling element 20 can be deployed onto the femoral nerve 342 and/or the sciatic nerve 344 in the leg 346, for example to treat post-operative knee or ankle pain and/or to treat osteoarthritis pain. The controller 330 can be located outside the body and/or implanted. The controller 330 can be in data and/or power communication with the cooling element(s) 20. The controller 330 can be adjusted manually and/or automatically. For example, if the patient senses more pain, the patient can increase the power on the controller 330, increasing the cooling (i.e., decreasing the temperature) of the cooling elements 20.

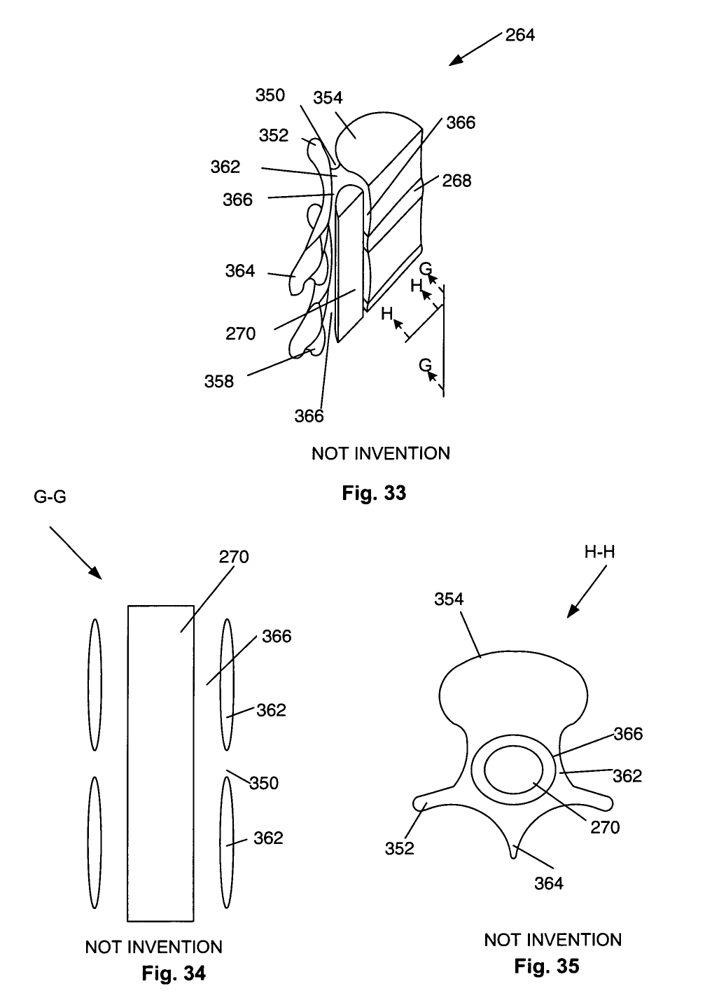

FIGS. 33 through 35 illustrate views of a length of the spinal column 264. Various anatomical features of the spinal column 348 are shown: intervertebral foramen 350, transverse processes 352, vertebral bodies 354, intervertebral discs 268, facets 358, the spinal cord 270, a pedicle 362, spinous processes 364, and the epidural space 366. The epidural space 366 is shown as open and expanded for clarity.

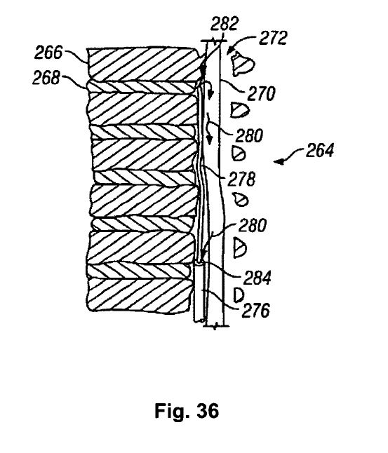

FIG. 36 shows the spinal column 264 through which a portion of spinal cord 270 is held within vertebral canal 272. Spinal column 264 is comprised of vertebrae 266 and intevertebral discs 268. A catheter 276 having inner tubing 278 slidably disposed within outer tubing 276 may be inserted within a lower region of vertebral canal 272. Inner tubing 278 may be advanced up within vertebral canal 272 while holding outer tubing 276 in position relative to the patient. Once inner tubing 278 has been desirably positioned upstream within the cerebrospinal fluid, a cooling fluid, as described above, may be pumped out of distal end 282 of inner tubing 278 such that it flows downstream with the cerebrospinal fluid, as shown by arrows 280, while cooling the spinal column 270 as the fluid flows. When the fluid has reached outer tubing 276 downstream, the fluid that has been warmed by the surrounding cerebrospinal fluid and tissue may be drawn into lumen 284 of outer tubing 276. This spent fluid may be recycled and reinjected through catheter 276. Spinal column 264 may be cooled along the entire length of the spinal column 264 or along portions of the length of the spinal column 264 by as little as, e.g., about 2° to about 3° C. below body temperature, for example to alleviate pain in a patient. The cooling fluid may be injected for as little as several minutes to as long as several hours at a time at an injection flow rate of, e.g., about 5 cc/min to about 10 cc/min, depending upon the particular condition of the patient.

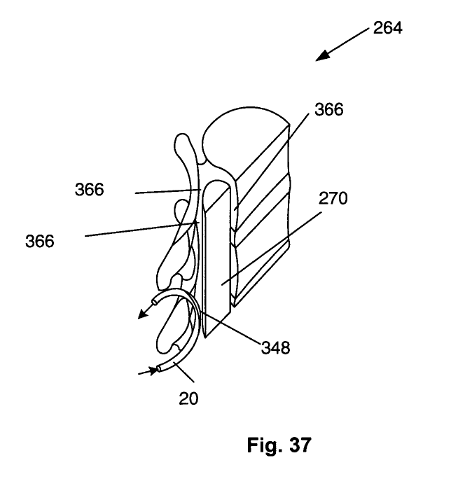

The cooling element 20 can be a single-lumen catheter. The cooling element 20 can be cooled along the entire length of the catheter 276. The catheter 276 can be inserted within the vertebral canal 272 to cool the spinal column 264. FIG. 37 illustrates that the cooling element 20 can deployed into, through, and out of the epidural space 366. The cooling element 20 can traverse the length of a single vertebra (as shown), two vertebrae, or more vertebrae. The fluid medium 58 can be pumped through the cooling element 20, as shown by arrows. The cooling element 20 can have a conductive panel 348. The conductive panel 348 can be positioned in contact or otherwise adjacent to the spinal cord 270. The conductive panel 348 can be more thermally conductive than the remainder of the cooling element 20.

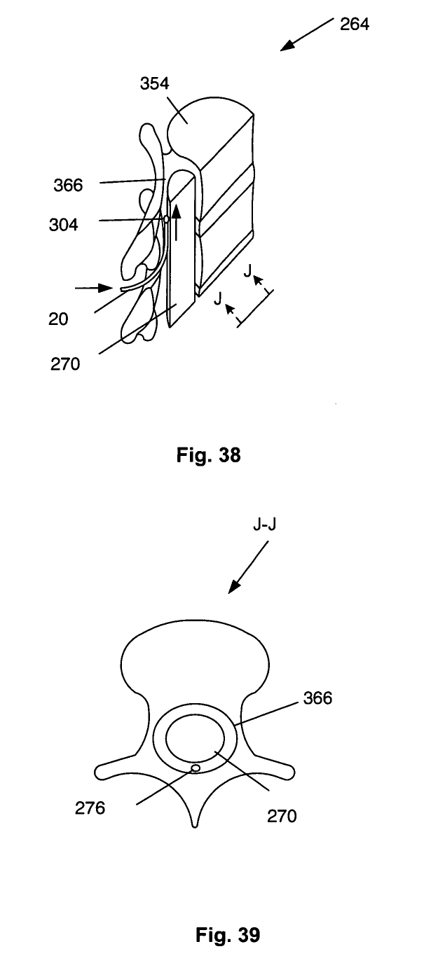

FIGS. 38 and 39 illustrates that the cooling element 20 (e.g., one of the embodiments shown in FIGS. 18-25) can be deployed into the epidural space 366, as shown by arrows. The cooling element 20 can be in the dorsal or ventral side of the epidural space 366. The cooling element 20 can be deployed in minimally invasively—or in an open procedure. The cooling element 20 can have radiopaque markers, for example to aid positioning the cooling element 20 during deployment. The tip balloon 304 can be advanced nominally beyond a target cooling site.

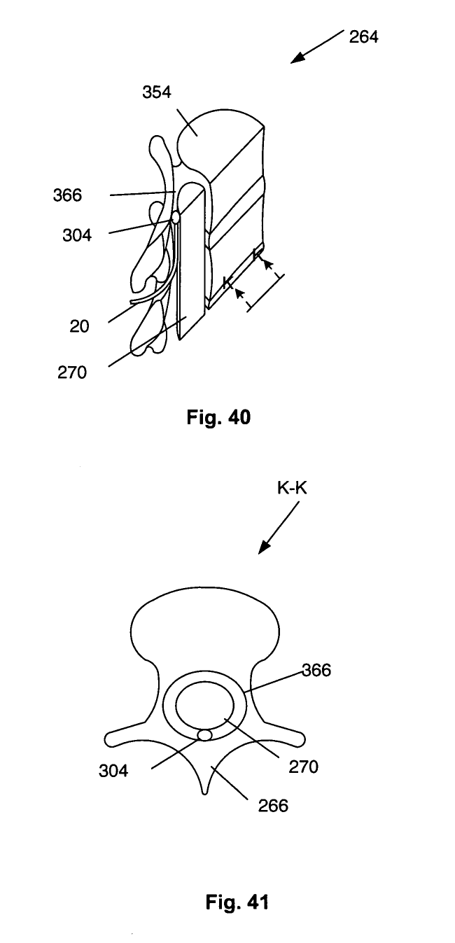

FIGS. 40 and 41 illustrate that the tip balloon 304 can be inflated. The tip balloon 304 can fix or anchor between the vertebra 266 and the spinal cord 270 (i.e., the dura). Other anchoring mechanisms (e.g., soft expandable arms, not shown) can be deployed.

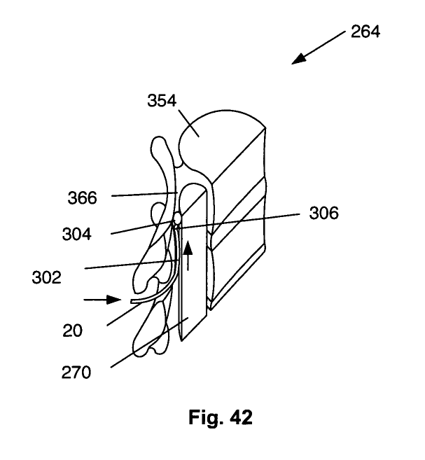

FIG. 42 illustrates that the cooling element 20 can be forced, as shown by arrows, into the epidural space 366 after the anchoring mechanism(s) is deployed (e.g., balloon tip inflation). The catheter neck 306 can offset or rotate from the longitudinal axis of the catheter body 302 adjacent to the catheter neck 306.

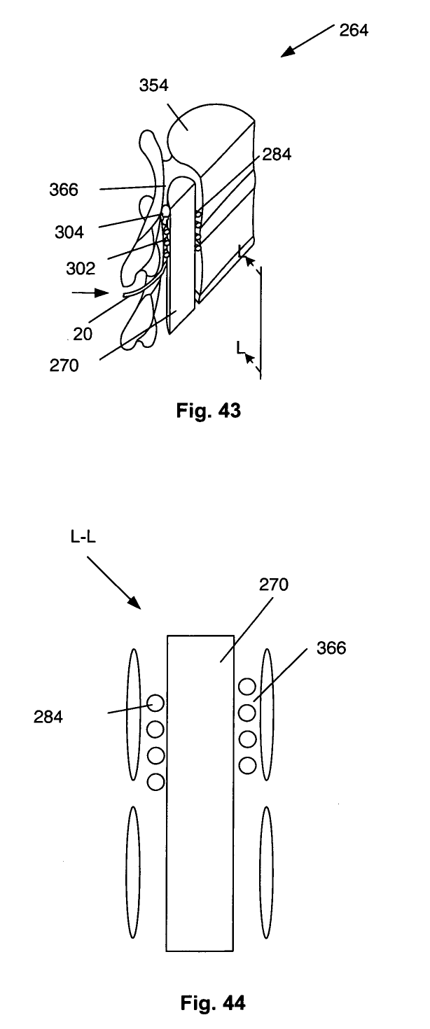

FIGS. 43 and 44 illustrate that the cooling element 20 can be forced into the epidural space 366. With the distal tip anchored between the vertebra 266 and the outer layer (i.e., dura) of the spinal cord 270, the catheter body 302 can translate into the epidural space 366. The catheter body 302 can circle the spinal cord 270 and/or fold upon itself (i.e., the catheter body 302). The catheter body 302 can densely pack around the complete or partial perimeter of the spinal cord 270 along a length of the spinal cord 270 adjacent to the tip balloon 304.

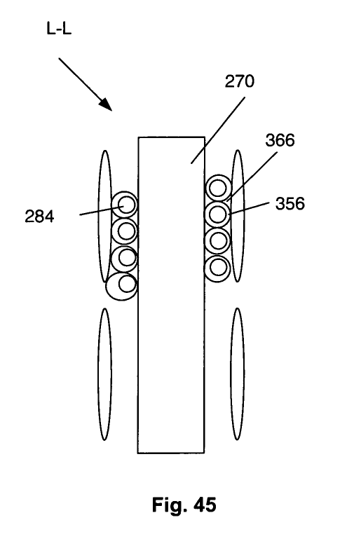

FIG. 45 illustrates that the body anchors can be deployed (e.g., body balloons 350 can be inflated). The body anchors can fix and/or anchor the cooling element 20 between the outer layer of the spinal cord 270 (i.e., dura) and the vertebrae 266.

Once deployed, with or without body anchors, the fluid medium 58 can be pumped through the cooling element 20. The fluid medium 58 can be cooled inside the body, for example, by a Peltier junction 42, and/or outside the body, for example, by passing the proximal end of the catheter body 302 through a cold water or cold saline bath or other refrigeration technique of the catheter body 302 and/or fluid medium 58. The fluid medium 58 can absorb heat as it passes through the epidural space 366 and reduce the temperature of the adjacent tissue, for example the dorsal and/or ventral columns of the spinal cord 270. The fluid medium 58 can pass through one or more body balloons 356 and/or the outer thermal fluid channel.

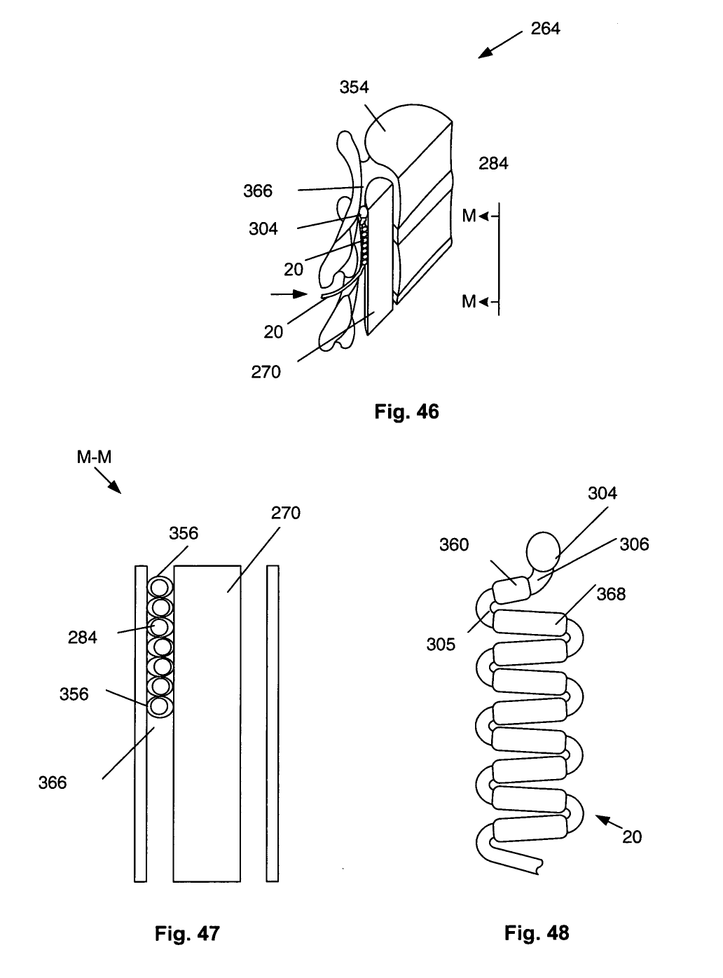

FIGS. 46 and 47 illustrate that the cooling element 20 can fold below itself and remain substantially on the dorsal side of the spinal cord 270. The cooling element 20 can cool the dorsal columns of the spinal cord 270. The cooling elements 20 can be deployed to not directly cool the ventral columns of the spinal cord 270. The cooling element 20 can be deployed onto only one side (e.g., dorsal) of the spinal cord 270, for example, by sequentially inflating the body balloons 356 to fix or anchor lengths of the cooling element 20 when located in a desired position. The cooling element 20 can then be steered (e.g., pushed or pulled) during deployment to achieve a desired configuration.

FIG. 48 is a dorsal view of the configuration of the deployed cooling element 20 from FIGS. 46 and 47 without the anatomical structures visible. The first body balloon 360 can be about half the length of the second 368 and other body balloons. The body balloons 360 can be substantially transverse to the longitudinal axis of the spinal cord 270. The catheter body 302 between each of the body balloons 356 can be configured in a curve of from about 130° to about 175°. The catheter neck 306 can be configured in a curve of from about 45° to about 85°.

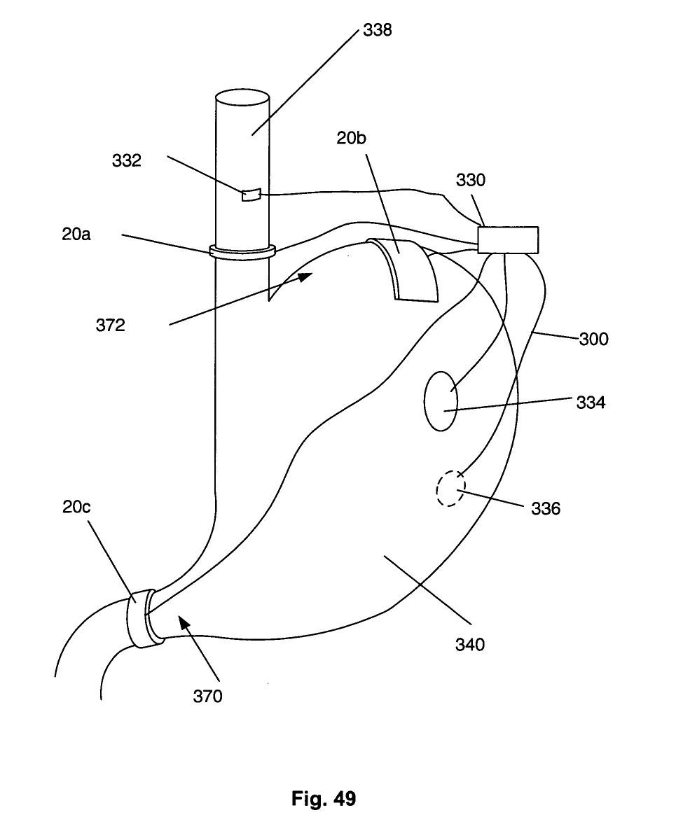

FIG. 49 illustrates that the cooling elements 20 can be attached to tissue, such as organs. The cooling elements 20a and 20c can be Peltier junctions 42 configured as cuffs. The cooling element 20a can be attached to the esophagus 338. The cooling element 20c can be attached to the pylorus 370. The cooling element 20b can be a Peltier junction 42 configured as a flexible or rigid sheet. The cooling element 20b can be attached to the stomach 340, for example, the fundus 372. The cooling element 20 can completely or partially surround the circumference of the stomach 340 or other organ. The controller 330 can be used with the various cooling elements 20a and/or 20b and/or 20c as described supra and in FIG. 31. The cooling element(s) can be attached to the heart, blood vessels (e.g., renal artery), intestines, one or more bones, uterus, kidney, liver, pancreas, lung, trachea, orbit, skin, testes, ovaries, vagina, eyes, ear canal, glands, lymph nodes, hair follicles, or combinations thereof. Moreover, any of the cooling element(s) can be attached to the underlying tissue utilizing any number of biocompatible attachment mechanisms or fasteners, e.g., barbs, hooks, sutures, bioadhesives, etc., or combinations thereof.

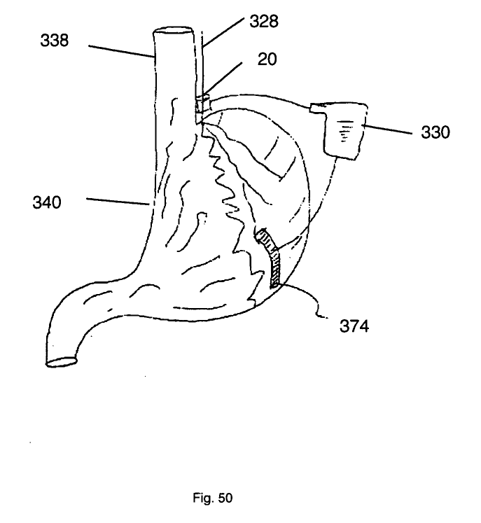

Further examples and variations of additional cooling systems may be seen in the following figures. For instance, one example shown in FIG. 50 illustrates a system where a strain gauge 374 (as described above) may be attached or adhered to the stomach surface 340 (for instance near or along the antrum of the stomach) via any number of attachment mechanisms, e.g., suturing, to the underlying muscularis layer of the stomach. The strain gauge 374 may be adhered to the outer serosal surface of the stomach 340, in which case delivery and deployment of the strain gauge 374 may be achieved via a laparoscopic or transgastric approach. Alternatively, the strain gauge 374 may be adhered to the inner mucosal surface of the stomach 340, in which case placement of the gauge 374 may be achieved via an endoscopic approach.

In either case, the strain gauge 374 may be calibrated to sense distension or movement of the stomach 340 which indicative of food ingestion. The strain gauge 374 may be connected via one or more wires (or wirelessly, as described above) to a controller 330, which may also be placed within the patient body, for instance, along the stomach 340 surface or to an intra-abdominal wall of the peritoneal cavity. The controller 330 may also be in electrical communication to the cooling element 20, which may be attached or adhered, e.g., the anterior vagus nerve trunk 328. When food has been ingested by the patient, the stomach 340 movement and/or distension may be sensed by the strain gauge 374 which then relays electrical signals to the controller 330. The controller 330 may then be configured to actuate the cooling element 20 appropriately to inhibit or altogether stop nerve transmission via cooling the vagus nerve trunk.

FIG. 51 illustrates another example of a cooling system in which the intragastric sensor 336 (described above), which may be placed against the serosal or mucosal surface and attached to the underlying muscularis layer, may be configured to wirelessly transmit signals to a receiver 378 on the controller 330. When the intragastric sensor 336 detects the presence of ingested food or liquid (or the presence of hydrochloric acid) within the stomach 340, it may transmit a wireless signal 376 to the controller 330, which may then activate the cooling element 20 or elements attached to the anterior 328 and/or posterior 326 vagus nerve trunks.

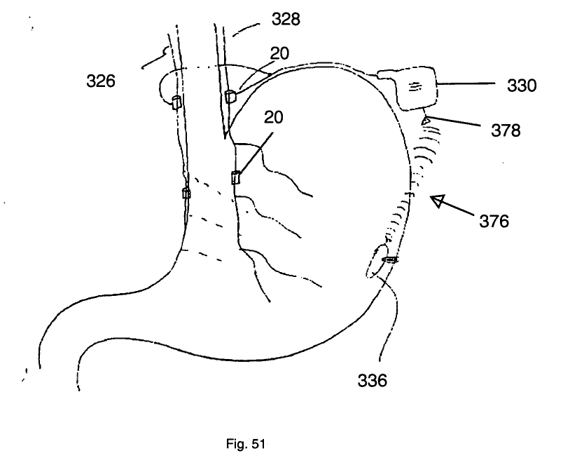

In yet another example as shown in FIG. 52A, an esophageal activation sensor 332 may be positioned around a portion of the esophagus 338, for instance, adjacent to the gastroesophageal junction either superior or inferior to the hiatus of the diaphragm. In either case, the esophageal activation sensor 332 may be configured as a circumferential or partially circumferential ring positioned about the esophagus 338 (as described above). The esophageal activation sensor 332 may be placed around the esophagus 338 either laparoscopically or transgastrically and it may include one or strain gauges 374 or pressure sensors positioned around the sensor.

When food or fluids are ingested and pass through the esophagus 338, the peristaltic movement and/or distension of the esophagus 338 as the food passes therethrough may be detected by the esophageal activation sensor 332. Signals correlating to the detected esophageal distension may be transmitted to the controller 330, which may then activate the one or more cooling elements 20 positioned upon or adjacent to the anterior 328 and/or posterior vagus nerve trunk 326. To reduce or eliminate the detection of false signals of esophageal distension (i.e., when no food is being ingested or passed into the stomach) from the esophageal sensor 332, the controller 330 may be configured to detect tissue distension, ΔD, beyond a threshold value for example, resulting in a distended esophageal diameter 380. The controller 330 may be configured to activate the one or more cooling elements if the number of instances of tissue distension, ΔD, over a predetermined time period, ΔT, is detected, as illustrated in FIG. 52B.

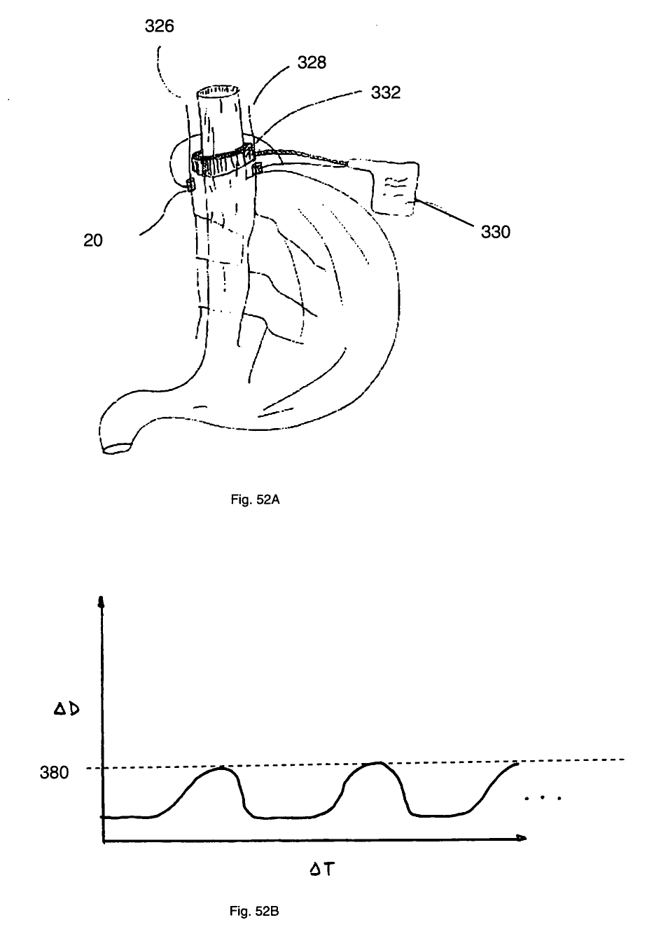

In yet another example, FIG. 53 shows an example of a system in which the cooling element 20 may be activated manually by the patient or another via an external remote 382. The patient, for instance, may signal the controller 330 to activate the cooling element 20 by transmitting a signal wirelessly 376 from outside the patient body to an antenna or receiver 378 integrated with the controller 330, e.g., prior to or during a patient's meal. The external remote 382 may be carried as a simple electronic controller or it may also be configured into any number of consumer devices, e.g., personal digital assistants, cellular phones, etc.

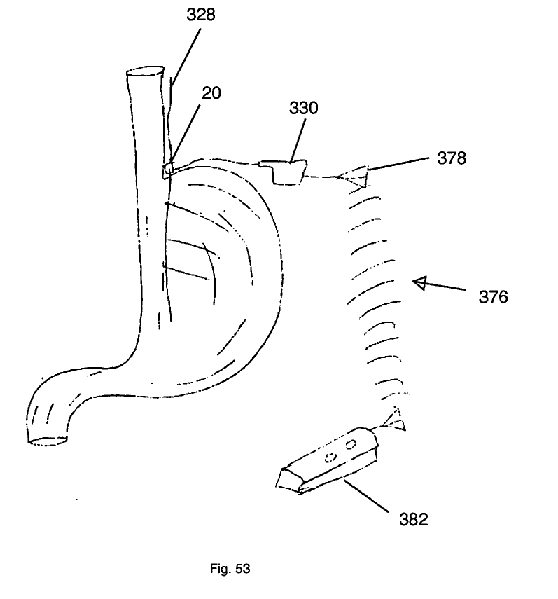

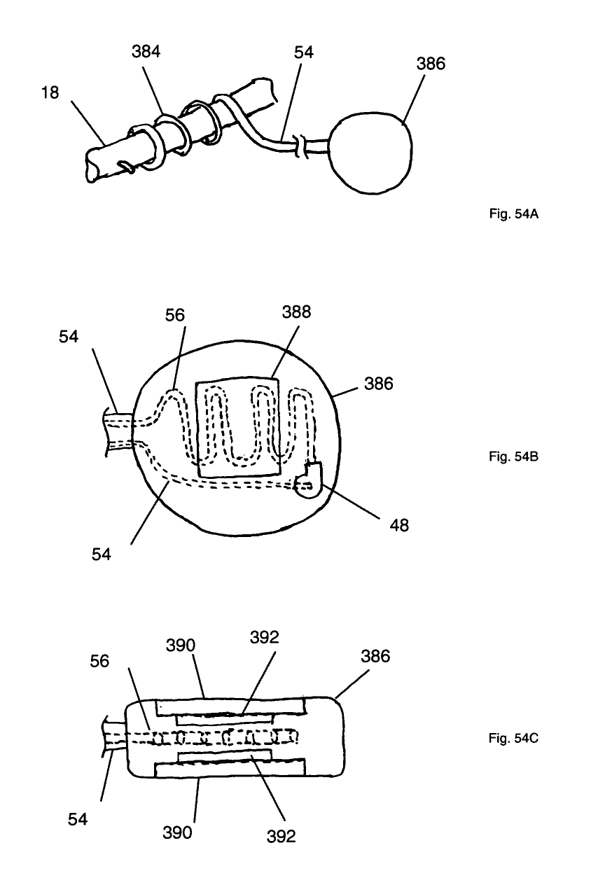

In another variation of the cooling system, as shown in FIG. 54A, the cooling element 20 may be configured into a helical cooling element 384 having a flexible and conformable length which may be wrapped at least partially around a nerve body 18. The helical cooling element 384 may have a cooling fluid or gas pumped through its coils to cryogenically cool the underlying nerve structure. The feedline 54 may connect the helical cooling element 384 to a controller and cooling unit 386. Examples of a cryogenic helical cooling element 384 may be seen further detail in U.S. Pat. Pub. 2003/0088240 A1 filed Dec. 5, 2001, which is incorporated herein by reference in its entirety.

The controller and cooling unit 386 is illustrated in FIG. 54B where the cooling fluid or gas may be pumped through the feedline 54 via at least one pump 48, which fluidly connects the coolant return line 56 with the coolant feedline 54. As the heated fluid is pumped through the return line 56, it may be snaked or coiled adjacent to or in thermal contact against one or more Peltier cooling elements 388 located on either or both sides of the coolant return line 56. As the heated fluid passes therethrough, it may be cooled or otherwise charged via the Peltier cooling elements 388 and then pumped back into the coolant feedline 54. As shown in FIG. 54C, the one or more Peltier coolers 392 may be in thermal contact with a corresponding heatsink 390 (made for example from Titanium), which may be conduct the thermal energy from the fluid and Peltier cooler 392 directly through the controller and cooling unit 386 case and subsequently into the surrounding tissue region.

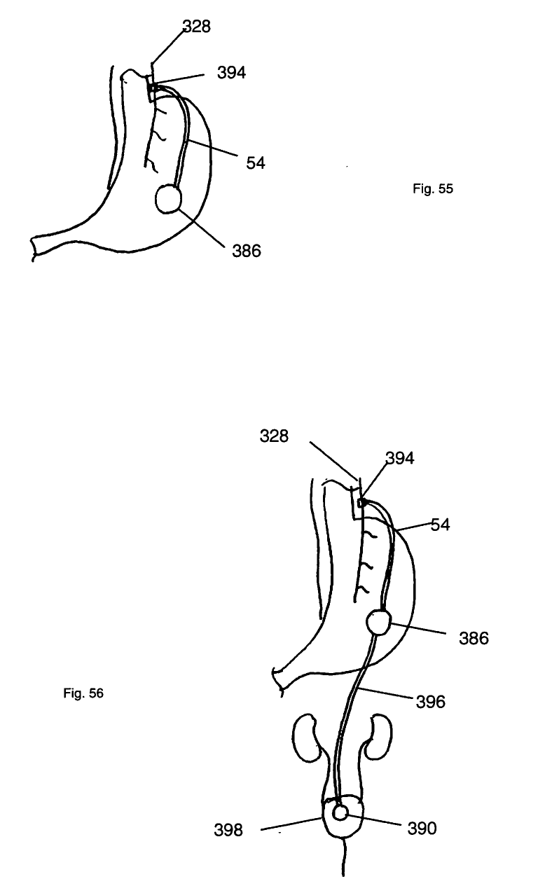

FIG. 55 shows an example where the device of FIG. 54A may have a cooling element 394, configured into the helical structure or otherwise, may be coupled fluidly to the controller and cooling unit 386, which in this example is attached to the stomach 340 surface. The controller and cooling unit 386 may conduct the heat through its one or more heatsinks 390 directly into the underlying serosal tissue of the stomach 340, which acts itself as a heatsink 390. Alternatively, as shown in FIG. 56, the controller and cooling unit 386 may be attached to the stomach 340 (or other body structure) and a separate or additional heatsink 390 unit may be attached via an additional cooling line 396 to yet another organ body such as the bladder 398, which has a relatively higher specific heat capacity when filled with urine and may act as an efficient heatsink 390 when urine, heated by the thermal energy from the attached heatsink 390, is voided from the patient body. In other examples, the heatsink 390 (or heatsinks) may be attached to other body structures such as the intra-abdominal wall of the peritoneal cavity, the uterus in females, etc.

As mentioned in the examples above, the controller and cooling unit 386 may be attached to the stomach 340 interior or exterior. If placed against the serosal tissue wall 402, one or more attachment mechanisms may be used to adhere the unit to the tissue. In the example of FIG. 57A, sutures 400 attached to the unit may be passed through the stomach wall, preferably through at least the muscularis layer 404 to ensure a secure and lasting attachment. If the unit is placed within the stomach interior against the mucosal layer 406, as shown in FIG. 57B, it may be attached again at least through the underlying muscularis layer 404 to ensure secure attachment. The feedline 54 passing through the stomach wall may be passed through a simple gastrotomy. The feedline 54 may have a porous member to facilitate the ingrowth of the surrounding tissue around the feedline 54. Alternatively, the feedline may be passed through an initial gastrotomy entry 408 within the mucosal layer 406 and then tunneled a short distance against the muscularis layer 402 to then exit through a gastrotomy exit 410 through the serosal layer 402. Including a tunneled portion of feedline 412 within the stomach tissue layers may help to inhibit the passage of food and fluids through the gastrotomy and into the peritoneal cavity of the patient.

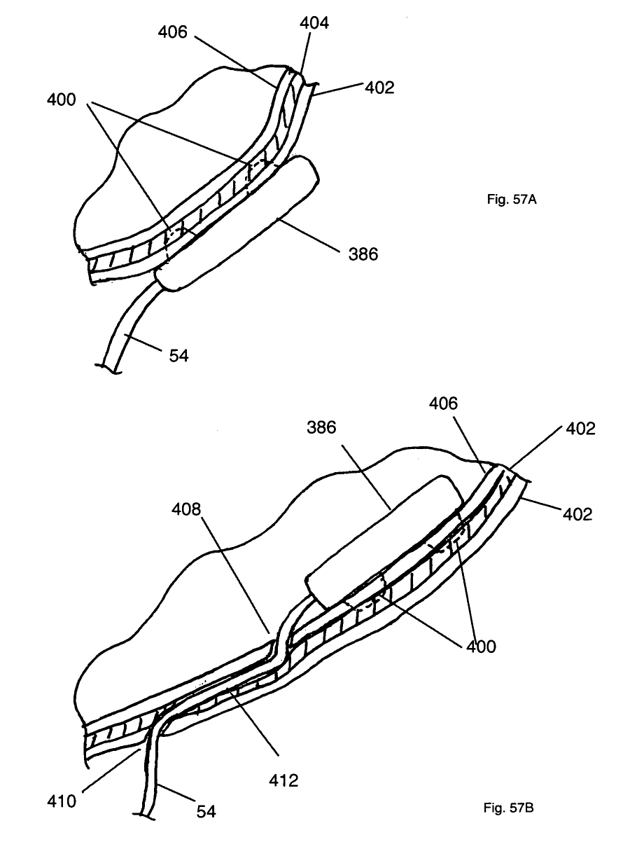

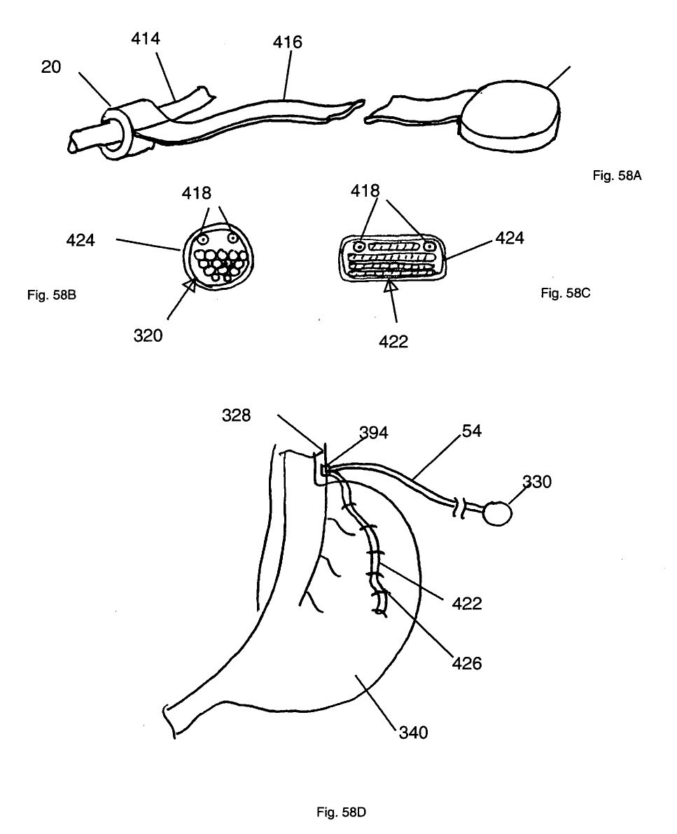

In yet another example of a cooling system, the cooling element 20 positioned against or around the nerve trunk 414 may be thermally coupled to the controller 330 via a thermal conduction line 416, as illustrated in FIG. 58A. The thermal conduction line 416 may include one or more power lines 418 connecting the controller 330 to the cooling element 20 and one or more conductive elements routed through the length of the conduction line to simply conduct the thermal energy generated by the cooling element 20 away from the nerve trunk 414 back towards the controller 330. As the thermal energy generated by the cooling element 20 is conducted away through the conduction line 416, this energy may be dissipated along the length of the conduction line 416 into the surrounding tissue structures either via radiative or conductive energy transfer.

FIG. 58B shows an example of a thermal conduction line 416 cross section to illustrate the use of one or more conductive cables 420. FIG. 58C shows another example where one or more conductive straps 422 may be positioned against one another. Any number of conductive materials may be used, e.g., copper, aluminum, titanium, etc. Multiple cables or straps may be utilized to increase the cross sectional heat transfer area while maintaining flexibility along the length of the conduction line as it is routed through the patient body. An insulative layer 424 surrounding the conduction line may be omitted entirely to utilize a thin conductive skin or it may be configured to vary in its thickness along the length of the conduction line to optimize the heat conduction away from the conduction line and into the surrounding tissue structures.

One example of use is shown in FIG. 58D where a conductive strap 422 may be thermally coupled to a cooling element 20. The conductive strap 422 may be attached via one or more attachment points 426, e.g., sutures, to a body structure such as the stomach. Thus, when the controller 330 activates the one or more cooling elements 20, the heat generated by the cooling element 20 may be conducted through the coupled conductive strap 422, which may then transfer the heat directly into the underlying tissue structure. Such a system may eliminate the need for pumps or cooling fluids.

The methods and apparatuses described herein can be used to rehabilitate from, treat or diagnose acute or chronic conditions and the pain resulting therefrom including multiple sclerosis (e.g., by cooling the spinal cord), chronic pain (e.g., by cooling the spinal cord and/or local nerves around the pain source), pancreatitis and/or pancreatic cancer (e.g., by cooling the celiac plexus), daily or migraine headaches including occipital neuralgia (e.g., by cooling the spinal cord around the C1-C2 vertebra, and/or the L2-3 or L3-4 vertebra, and/or by cooling the occipital nerve), post-operative pain relief (e.g., by cooling nerve(s) near the surgical site, cooling the spinal cord, or such as shown and described by FIG. 32 for orthopedic knee surgery), interstitial cystitis (e.g., by cooling the bladder tissue such as with a band around the bladder), osteoarthritis or other local joint pain (e.g., by cooling the local nerves, cooling the spinal cord, or as shown and described by FIG. 32 for knee arthritis or other knee pain), obesity (e.g., by cooling the vagus nerve and branches and trunks thereof, such as shown and described by FIG. 31), cancer (e.g., by cooling—such as freezing—the ganglion of Impar), plantar fasciitis, congestive heart failure (CHF), facial pain (e.g., by cooling facial nerves), cervical dystonia, chronic pelvic pain (e.g., by cooling the ganglion of Impar), Parkinson's and/or Alzheimer's disease (e.g., by cooling selective locations within the brain). The methods and apparatuses described herein can also be used for localized cryoablation and localized ablation.

During use of the methods and apparatus described herein, the target nervous system tissue can be cooled to a nerve tissue temperature from about 15° C. to about 37.5° C., for example, from about 20° C. to about 35° C., for example about 20° C.

All of the controllers and controlling electronics disclosed herein can have processors, such as microprocessors known to one having ordinary skill in the art.

The applications of the cooling devices and methods discussed above are not limited to fibrous nerve bodies, regions within the brain, or regions of the spinal cord but may include any number of further treatment applications. Other treatment sites may include areas or regions of the body such as organ bodies.

Any elements described herein as singular can be pluralized (i.e., anything described as “one” can be more than one). Any species element (e.g., body balloon) of a genus element (e.g., body anchor) can have the characteristics or elements of any other species element of that genus. The above-described configurations, elements or complete assemblies and methods and their elements for carrying out the invention, and variations of aspects of the invention can be combined and modified with each other in any combination.

We claim:

1. A tissue temperature alteration apparatus comprising:
a controller;
a cooling element in data communication with the processor; and
a digestive activation sensor in data communication with the controller;
wherein the controller is configured to activate the cooling element when the digestive activation sensor transmits an activation data to the controller.

2. The apparatus of claim 1, wherein the digestive activation sensor comprises a stomach sensor.

3. The apparatus of claim 2, wherein the stomach sensor comprises an intragastic sensor.

4. The apparatus of claim 2, wherein the stomach sensor comprises a stomach surface sensor.

5. The apparatus of claim 1, wherein the activation sensor comprises an esophageal activation sensor.

6. The apparatus of claim 1, wherein the controller comprises a processor.

7. A tissue temperature alteration device comprising:
An elongated body having a distal end;
an anchoring mechanism on the distal end of the elongated body;
a first channel along the elongated body;
a second channel along the elongated body, wherein the first channel is in fluid communication with the second channel at the distal end; and

8. The device of claim 7, further comprising a third channel, wherein the third channel is in communication with the anchoring mechanism.

9. The device of claim 7, wherein the first channel is radially outside of the second channel.

10. The device of claim 7, wherein the anchoring mechanism is a balloon.

11. The device of claim 7, wherein the anchoring mechanism comprises radially extending arms.

12. The device of claim 7, wherein the third channel is in fluid communication with the anchoring mechanism.

13. The device of claim 7, wherein the third channel is in electrical communication with the anchoring mechanism.

14. The device of claim 13, further comprising a conductive wire in the third channel.

15. The device of claim 7, wherein the elongated body is resiliently deformable.

16. The device of claim 15, wherein the elongated body comprises a shape memory material.

17. The device of claim 7, wherein the elongated body is formed into a coiled configuration

18. The device of claim 17, wherein the elongated body is resiliently deformable.

19. The device of claim 18, wherein the elongated coil body comprises a shape memory material.

20. A method of deploying a heat transfer element into the epidural space comprising:
anchoring the heat transfer element in the epidural space;
advancing the heat transfer element into the epidural space such that the heat transfer element bends in a first direction; and
flowing a fluid through the heat transfer element.

21. The method of claim 20, further comprising cooling the fluid.

22. The method of claim 20, further comprising heating the fluid.

23. The method of claim 20, further comprising additionally advancing the heat transfer element into the epidural space so that the heat transfer element bends in a second direction.

24. The method of claim 23, wherein the heat transfer element comprises a first body anchor and the method further comprises deploying the first body anchor.

25. The method of claim 24, further comprising additionally advancing the heat transfer element into the epidural space so that the heat transfer element bends in the first direction.

26. The method of claim 25, wherein the heat transfer element comprises a second body anchor and the method further comprises deploying the second body anchor.

27. A method of local pain relief from a first nerve comprising:
implanting a heat transfer element adjacent to the first nerve, wherein the heat transfer element comprises a Peltier junction; and
controlling the heat transfer of the heat transfer element.

28. The method of claim 27, wherein the first nerve is the femoral nerve.

29. A method of treating multiple sclerosis by cooling the spinal cord comprising:
deploying a heat transfer element to the epidural space.

30. The method of claim 29, wherein deploying comprises advancing a catheter body into the epidural space.

31. The method of claim 30, wherein advancing a catheter body further comprises curving the catheter body in a first direction at a first length in the epidural space, and curving the catheter body in a second direction at a second length in the epidural space.

32. The method of claim 29, further comprising flowing cold fluid through the catheter body.

33. A method of minimally invasive deployment of a heat transfer element adjacent to a nerve, wherein the heat transfer element has a curved relaxed configuration, comprising:
applying a straightening force on the heat transfer element;
advancing the heat transfer element adjacent to the nerve; and
removing the straightening force from the heat transfer element.
